(19) United States
(12) Patent Application Publication
(10) Pub. No.: US 2023/0212181 A1
(43) Pub. Date: Jul. 6, 2023

(54) DOUBLE PAYLOAD CANCER THERAPEUTICS

(71) Applicant: Aarvik Therapeutics, Inc., Hayward, CA (US)

(72) Inventors: Vasu JAMMALAMADAKA, Dublin, CA (US); Jagath Reddy JUNUTULA, Fremont, CA (US)

(21) Appl. No.: 18/089,493

(22) Filed: Dec. 27, 2022

Related U.S. Application Data
(60) Provisional application No. 63/266,326, filed on Jan. 1, 2022.

(51) Int. Cl. C07D 491/147; A61K 47/68; C07D 519/00

(52) U.S. Cl. CPC C07D 491/147; A61K 47/6851; A61K 47/6803; A61K 47/6889; C07D 519/00; A61K 2039/505

(57) ABSTRACT

Disclosed herein are double payload antibody-drug conjugates (ADCs) and compositions comprising ADCs and one or more pharmaceutically acceptable carriers. Also, disclosed herein are methods for using these ADCs and compositions comprising ADCs and one or more pharmaceutically acceptable carriers to treat various cancers. Also disclosed herein are kits comprising compositions comprising ADCs.

### REFERENCE TO RELATED APPLICATIONS

This application claims the benefit of the filing date of U.S. provisional application 63/266,326, filed Jan. 1, 2022, the contents of which are incorporated in their entirely by reference.

### STATEMENT AS TO FEDERALLY SPONSORED RESEARCH

None.

### BACKGROUND

Antibody Drug Conjugates (ADCs) for use in cancer therapy combine the specificity of an antibody with the potency of a cytotoxic chemical. Each ADC comprises three components: (a) an antibody targeting an extracellular antigen expressed in tumor tissues; (b) a linker conjugated at one end to a native or engineered amino acid residue in the antibody, and (c) a cytotoxic payload conjugated to the other end of the linker.

After the ADC binds the antigen, the complex is internalized. Linkers can be designed to remain un-cleaved from the payload until the antibody itself is proteolytically degraded, or be cleaved by low pH, a redox partner, or an enzyme to release the payload. Various payloads have been tested in humans, but payloads on ADCs approved by the FDA include DNA-damaging agents (calicheamicin and tesirine), microtubule disrupting agents (Maytansine, MMAE, and MMAF), and topoisomerase-I inhibitors (deruxtecan and SN38).

Though ADCs were produced in the early 1970s (Ghose, T., et al., Cancer, 1972, 5:1398-1400), they first entered clinical testing in 1997. Some ADCs, such trastuzumab deruxtecan (Enhertu®), have been successful in treating human cancers (Cristcitiello, C., et al., J. Hematol. Oncol., 2021, 14:20). However, a far greater number have failed clinical trials. In the two dozen years since 1987, of the over 200 ADCs tested, only eleven have been approved by the U.S. Food and Drug Administration to date, while 88 have either been discontinued or had no updates publicly posted for the past three or more years. The clinical failure or limited success of many ADCs has been attributed to two reasons: insufficient efficacy, and/or intolerable toxicity.

While ADCs hold immense potential owing to the specific targeting of cytotoxic payloads to select tissues while sparing normal cells, off-target toxicity due to random release of the payload, and poor targeting to cancer cells not expressing a very high level of antigen or poor internalization of the antigen-ADC complex has severely curtailed the success of ADCs.

### SUMMARY

In one aspect provided herein is a compound of Formula (I):

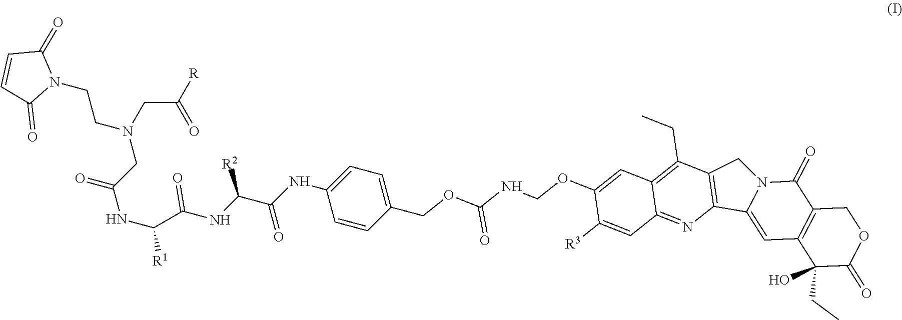

wherein:

R is selected from the group consisting of Formula (II), Formula (III), and Formula (IV):

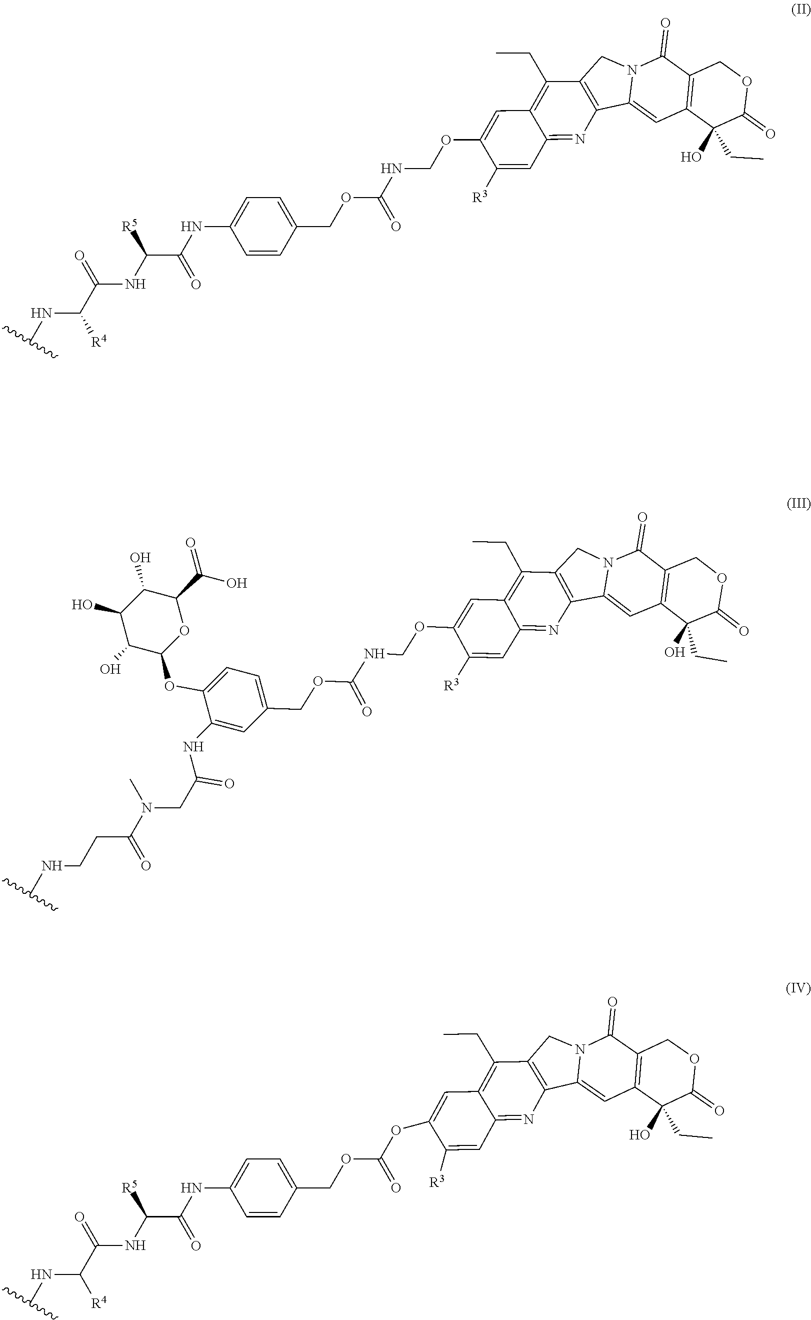

wherein:

R¹ is —CH₃ or —CH(CH₃)₂;

R² is —CH₃, —(CH₂)₃NHC(═O)NH₂ or —CH(CH₃)₂;

R³ is independently selected from H and F (this is, both the same or different);

R⁴ is —H, —CH₃ or —CH(CH₃)₂; and

R⁵ is —CH₃, —(CH₂)₄NH₂, —(CH₂)₃NHC(═O)NH₂ or —CH(CH₃)₂;

or a pharmaceutically acceptable salt thereof.

In one embodiment, the compound is selected from the group consisting of:

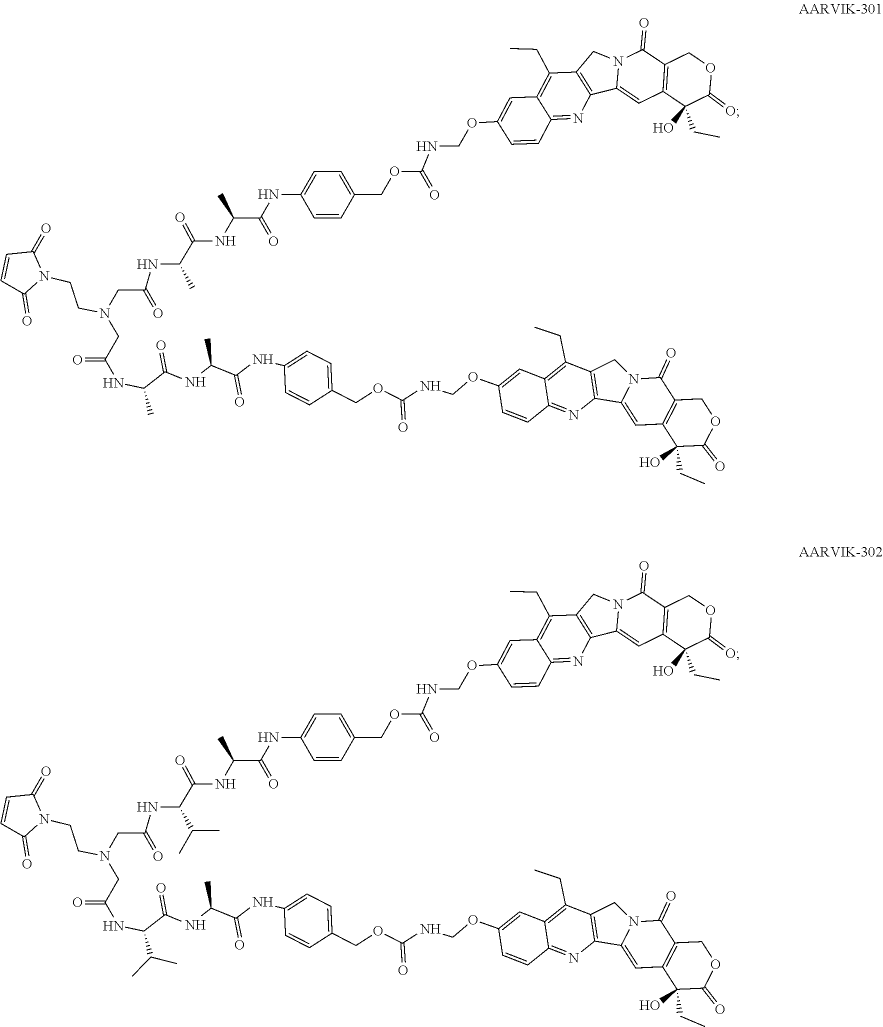 [SMILES: CCc1c2c(nc3ccc(OCNC(=O)OCc4ccc(NC(=O)[C@H](C)NC(=O)[C@H](C)NC(=O)CN(CCN5C(=O)C=CC5=O)CC(=O)N[C@@H](C)C(=O)N[C@@H](C)C(=O)Nc5ccc(COC(=O)NCOc6ccc7nc8c(c(CC)c7c6)Cn6c-8cc7c(c6=O)COC(=O)[C@]7(O)CC)cc5)cc4)cc13)-c1cc3c(c(=O)n1C2)COC(=O)[C@]3(O)CC ; CCc1c2c(nc3ccc(OCNC(=O)OCc4ccc(NC(=O)[C@H](C)NC(=O)[C@@H](NC(=O)CN(CCN5C(=O)C=CC5=O)CC(=O)N[C@H](C(=O)N[C@@H](C)C(=O)Nc5ccc(COC(=O)NCOc6ccc7nc8c(c(CC)c7c6)Cn6c-8cc7c(c6=O)COC(=O)[C@]7(O)CC)cc5)C(C)C)C(C)C)cc4)cc13)-c1cc3c(c(=O)n1C2)COC(=O)[C@]3(O)CC]

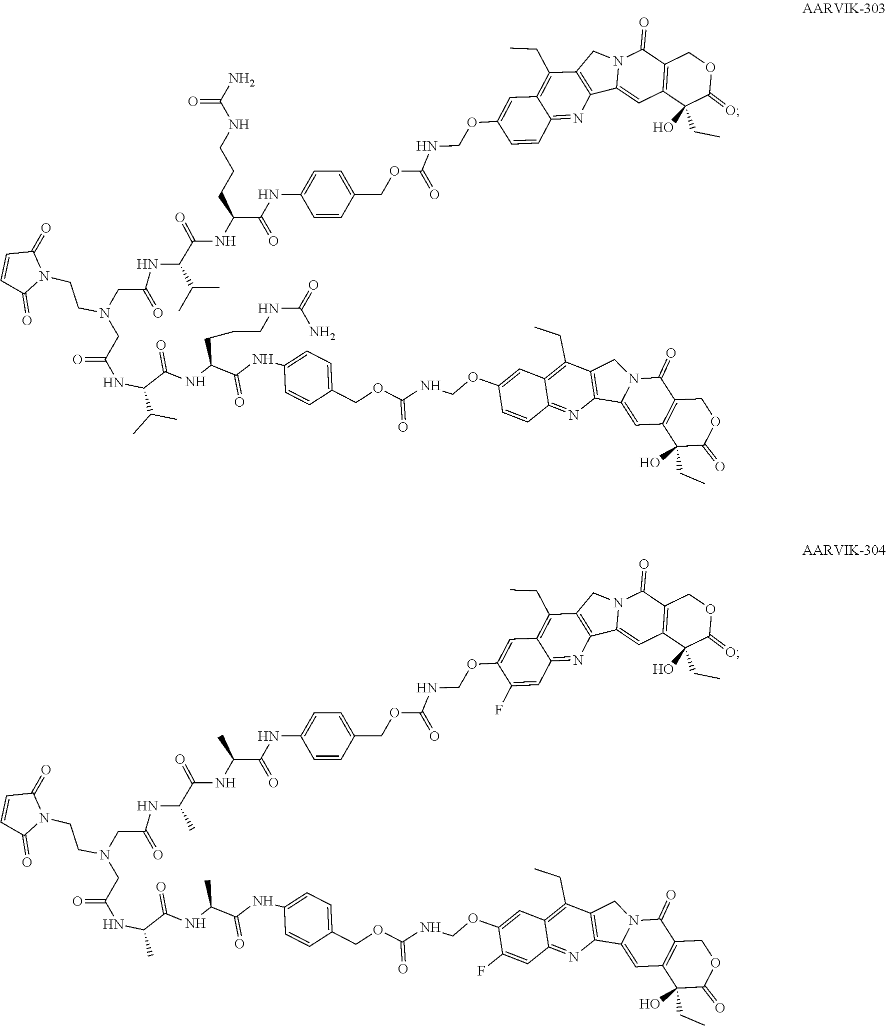

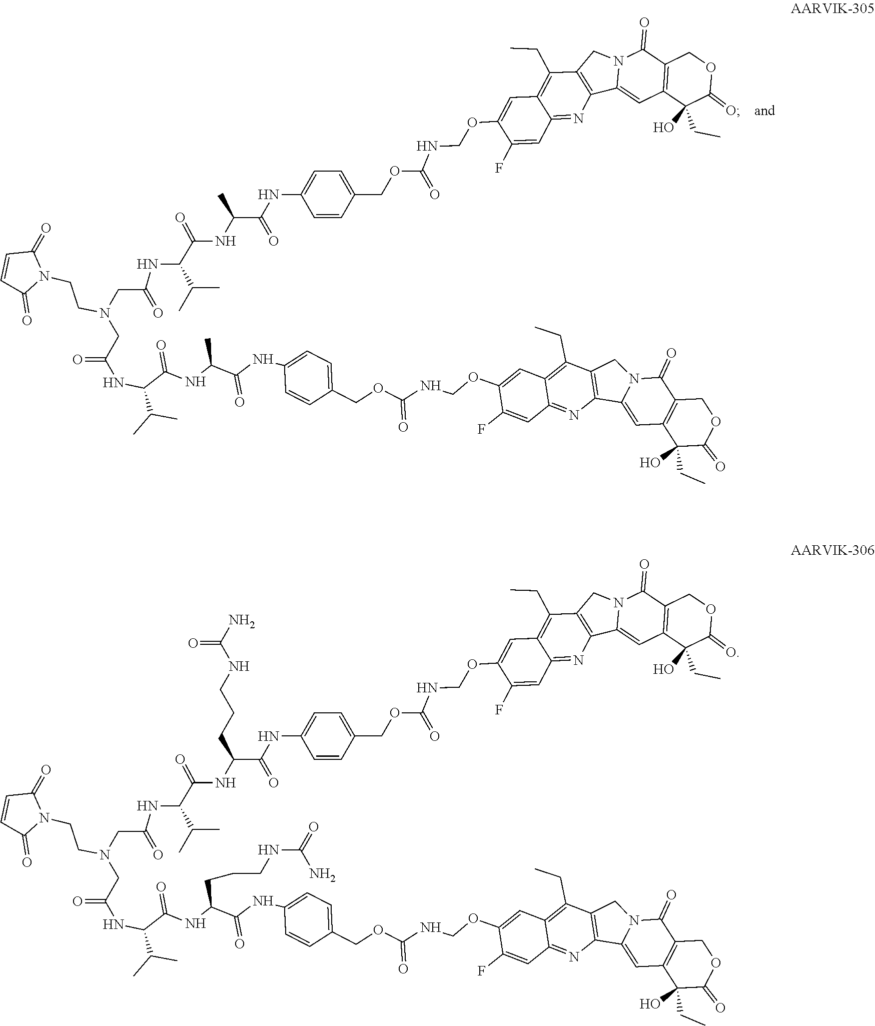

In another embodiment, the compound is selected from the group consisting of selected from the group consisting of:

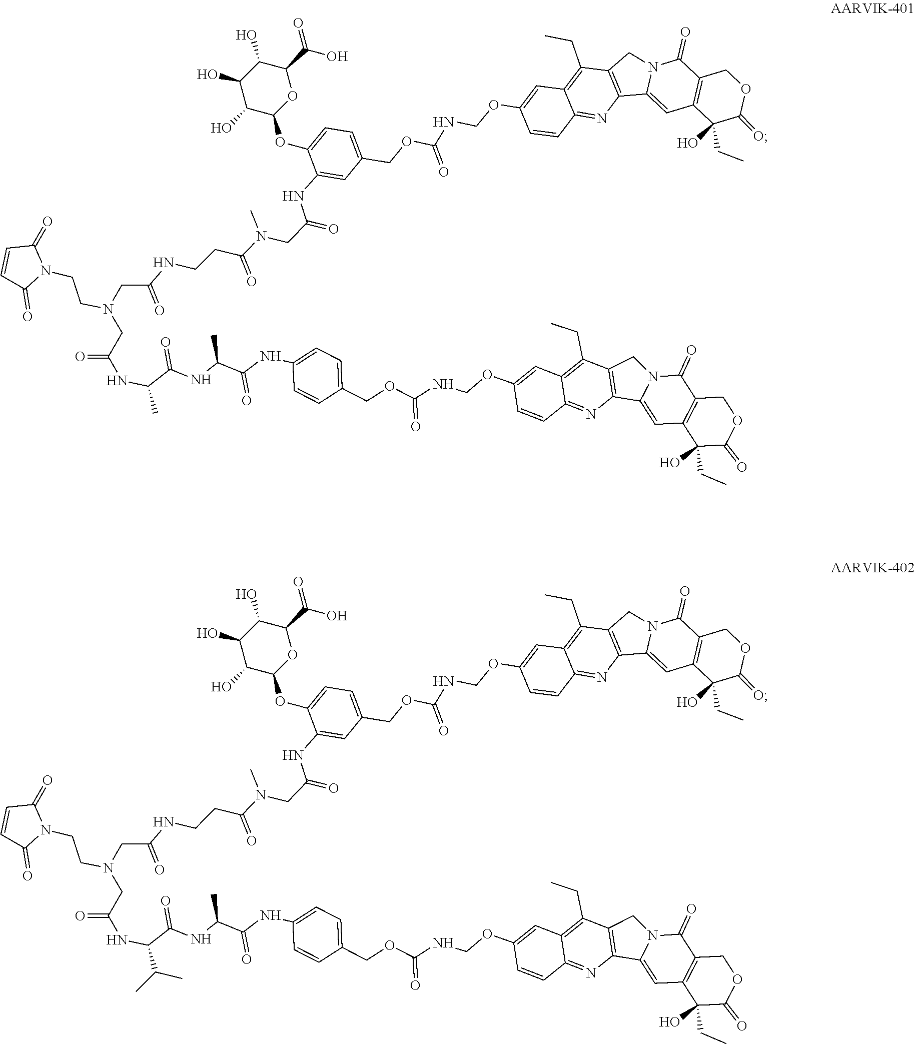

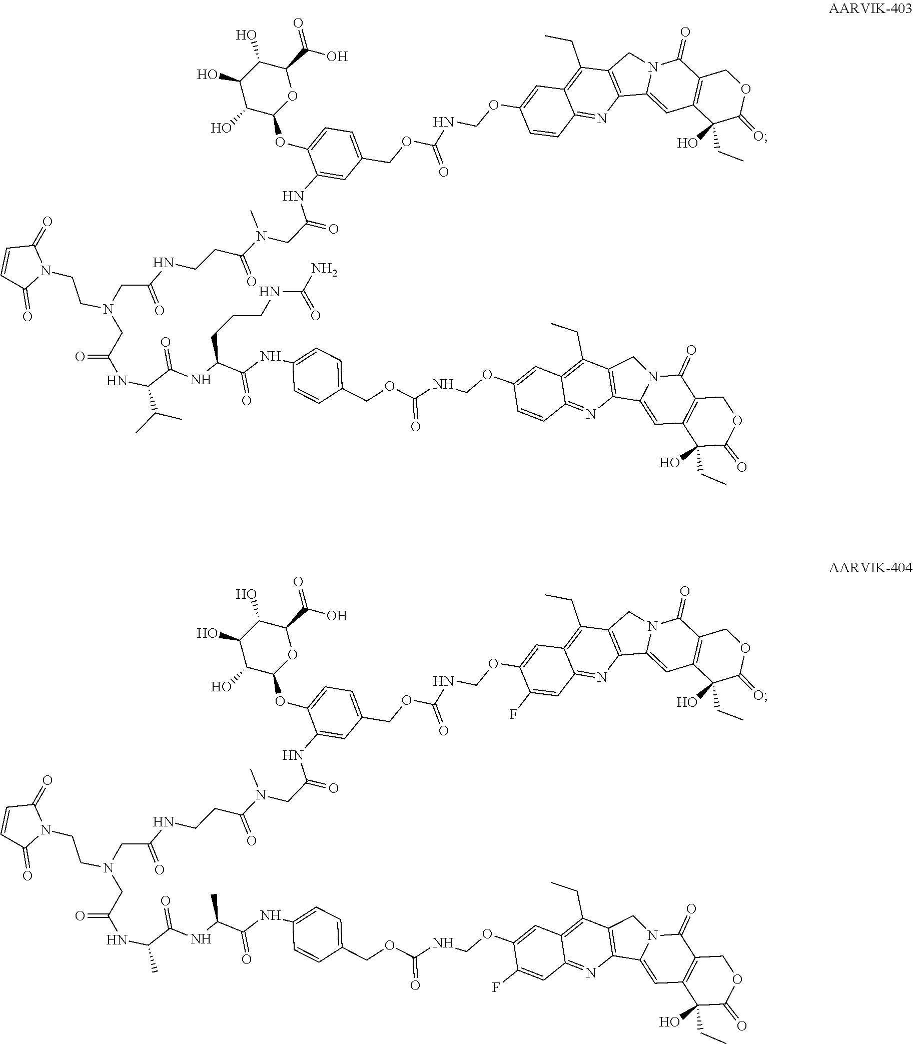

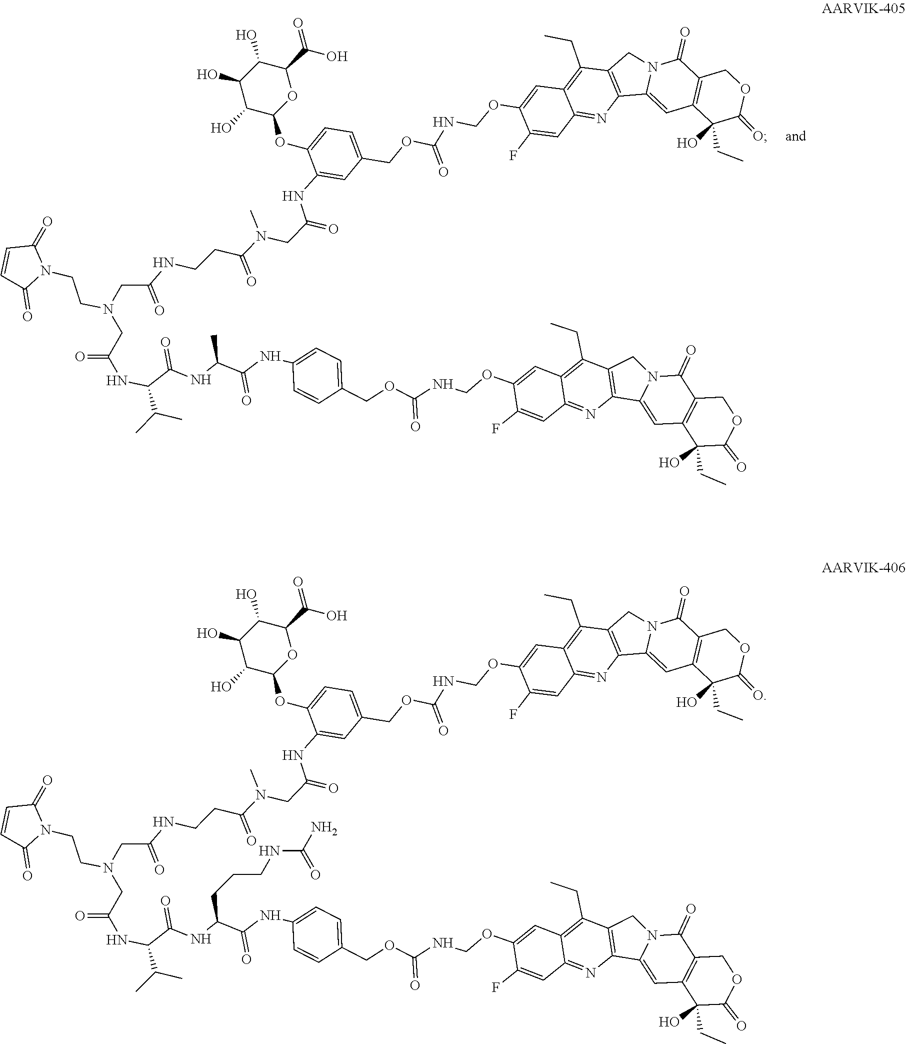

In another embodiment, the compound is selected from the group consisting of:

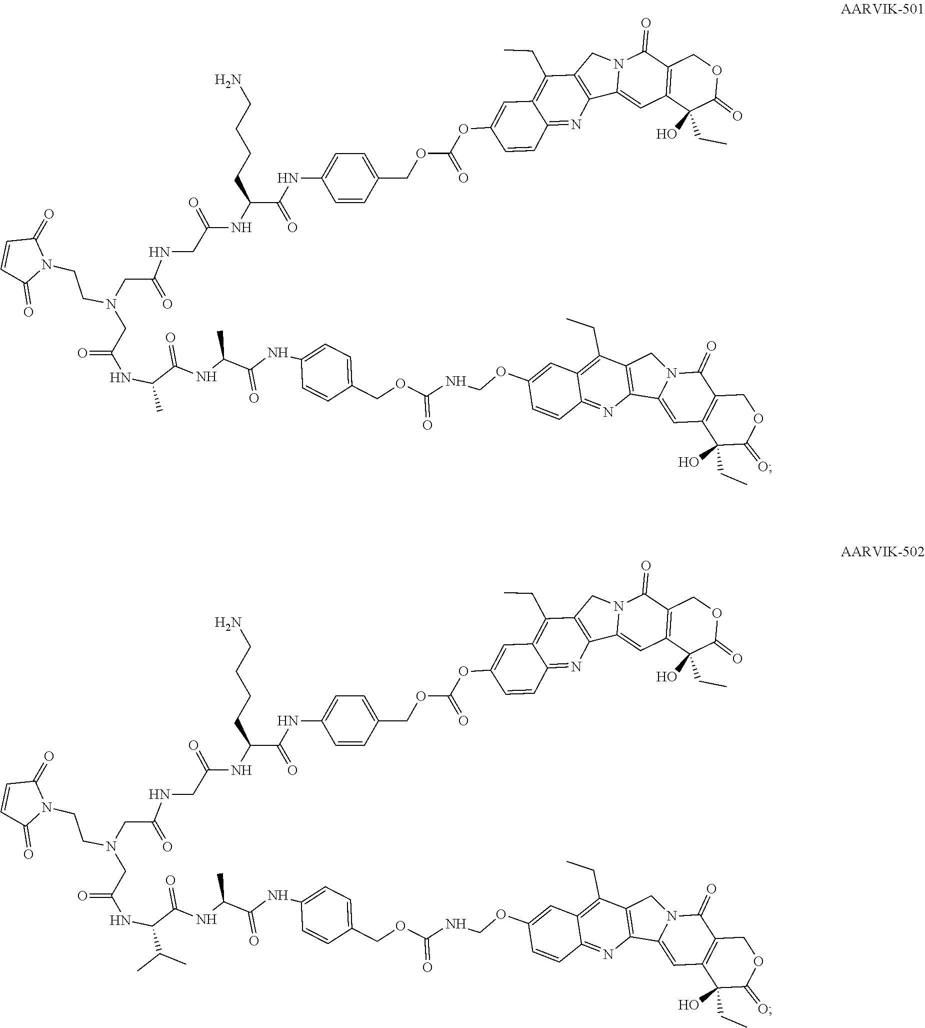 [SMILES: CCc1c2c(nc3ccc(OCNC(=O)OCc4ccc(NC(=O)[C@H](C)NC(=O)[C@H](C)NC(=O)CN(CCN5C(=O)C=CC5=O)CC(=O)NCC(=O)N[C@@H](CCCCN)C(=O)Nc5ccc(COC(=O)Oc6ccc7nc8c(c(CC)c7c6)Cn6c-8cc7c(c6=O)COC(=O)[C@]7(O)CC)cc5)cc4)cc13)-c1cc3c(c(=O)n1C2)COC(=O)[C@]3(O)CC ; CCc1c2c(nc3ccc(OCNC(=O)OCc4ccc(NC(=O)[C@H](C)NC(=O)[C@@H](NC(=O)CN(CCN5C(=O)C=CC5=O)CC(=O)NCC(=O)N[C@@H](CCCCN)C(=O)Nc5ccc(COC(=O)Oc6ccc7nc8c(c(CC)c7c6)Cn6c-8cc7c(c6=O)COC(=O)[C@]7(O)CC)cc5)C(C)C)cc4)cc13)-c1cc3c(c(=O)n1C2)COC(=O)[C@]3(O)CC]

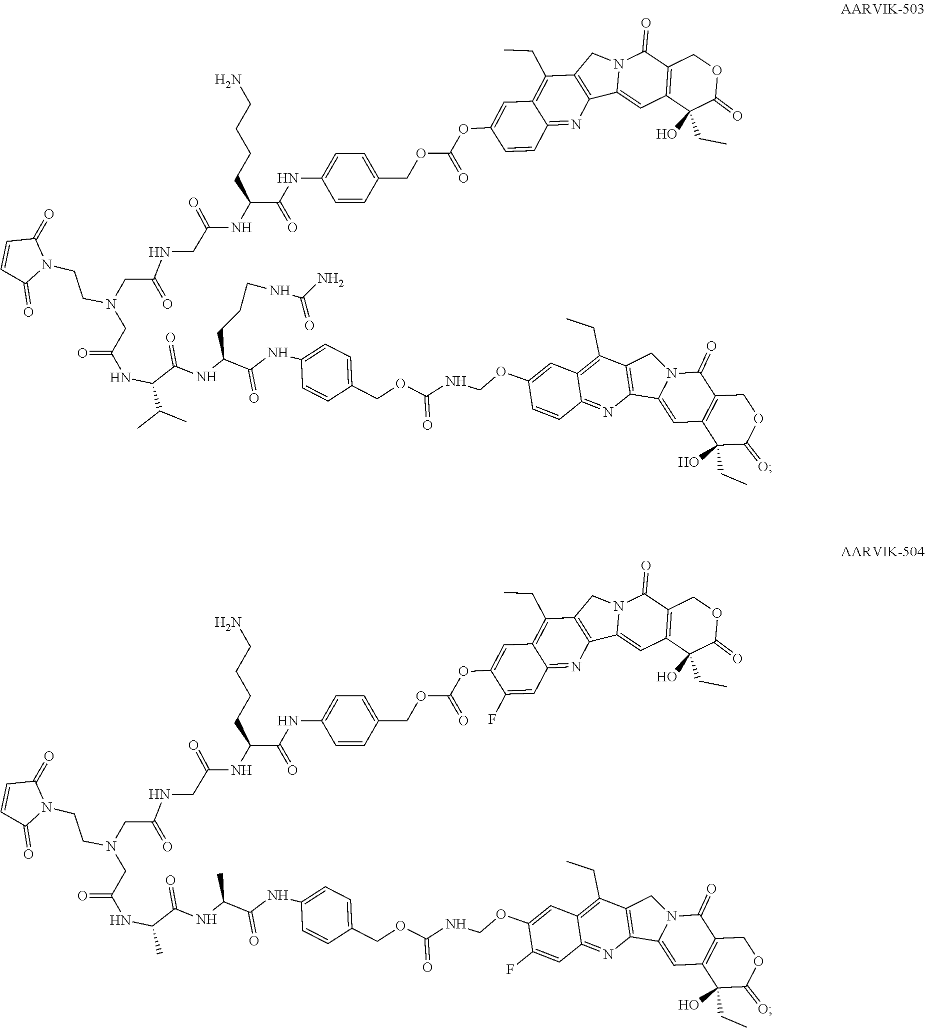 [SMILES: CCc1c2c(nc3ccc(OCNC(=O)OCc4ccc(NC(=O)[C@H](CCCNC(N)=O)NC(=O)[C@@H](NC(=O)CN(CCN5C(=O)C=CC5=O)CC(=O)NCC(=O)N[C@@H](CCCCN)C(=O)Nc5ccc(COC(=O)Oc6ccc7nc8c(c(CC)c7c6)Cn6c-8cc7c(c6=O)COC(=O)[C@]7(O)CC)cc5)C(C)C)cc4)cc13)-c1cc3c(c(=O)n1C2)COC(=O)[C@]3(O)CC ; CCc1c2c(nc3cc(F)c(OCNC(=O)OCc4ccc(NC(=O)[C@H](C)NC(=O)[C@H](C)NC(=O)CN(CCN5C(=O)C=CC5=O)CC(=O)NCC(=O)N[C@@H](CCCCN)C(=O)Nc5ccc(COC(=O)Oc6cc7c(CC)c8c(nc7cc6F)-c6cc7c(c(=O)n6C8)COC(=O)[C@]7(O)CC)cc5)cc4)cc13)-c1cc3c(c(=O)n1C2)COC(=O)[C@]3(O)CC]

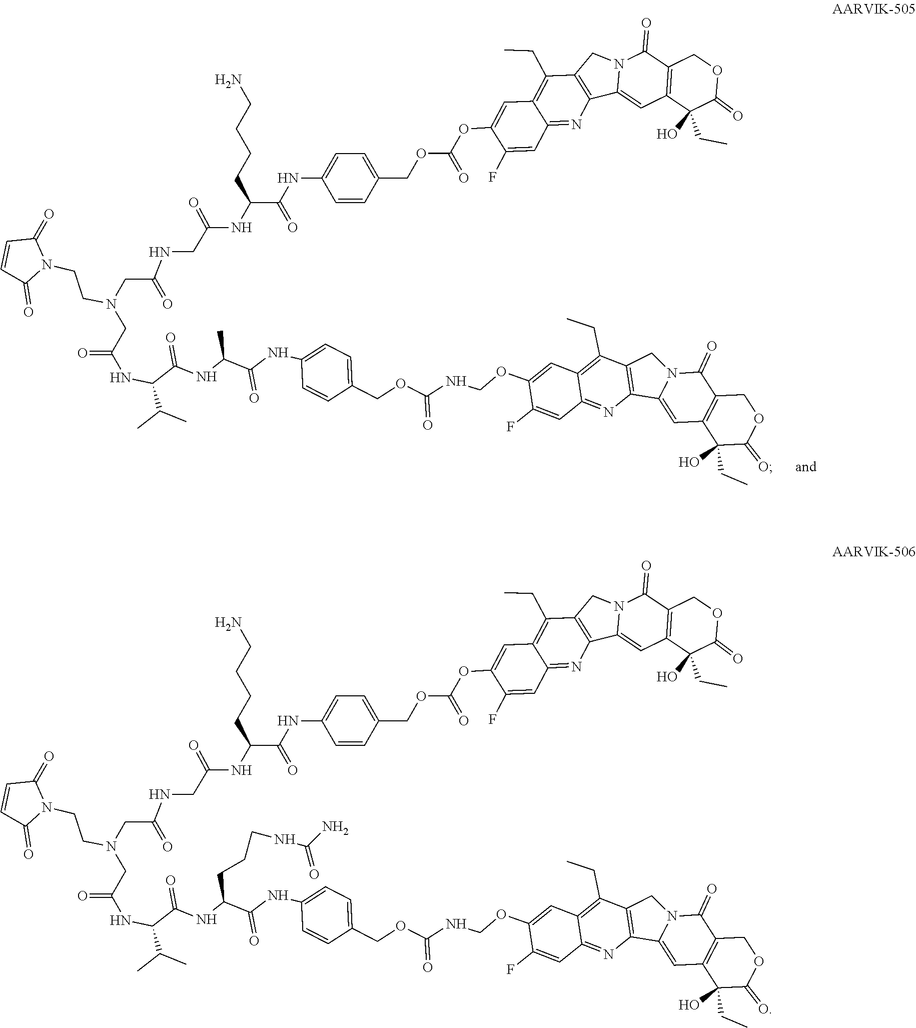

In another aspect, provided herein in a compound of Formula (V):

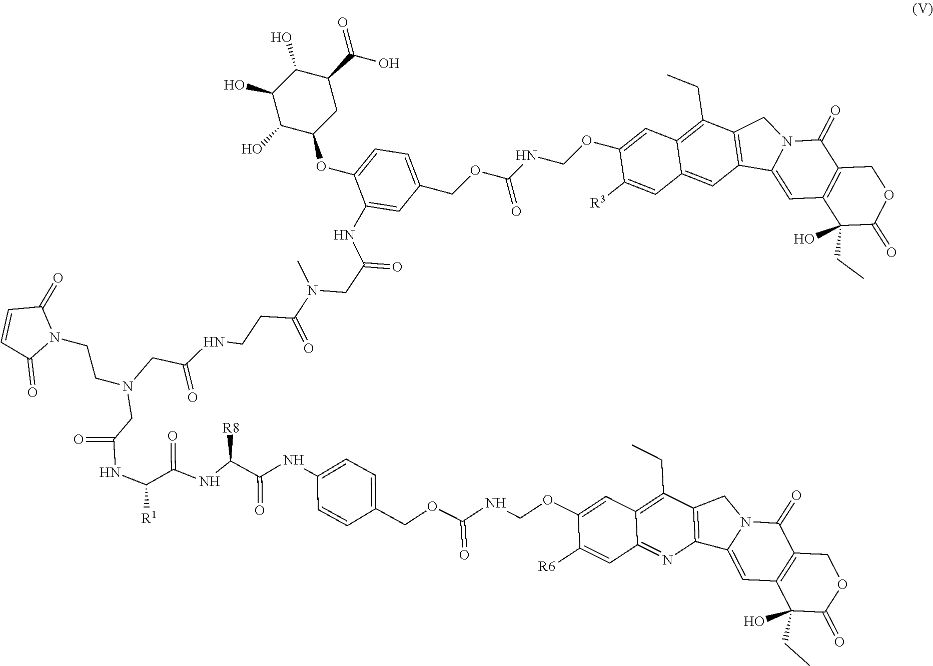

wherein:

R⁶ is H or F;

R⁷ is —H, —CH₃ or —CH(CH₃)₂;

R⁸ is —CH₃, —(CH₂)₄NH₂, —(CH₂)₃NHC(═O)NH₂ or —CH(CH₃)₂;

or a pharmaceutically acceptable salt thereof.

In one embodiment, the compound is selected from the group consisting of:

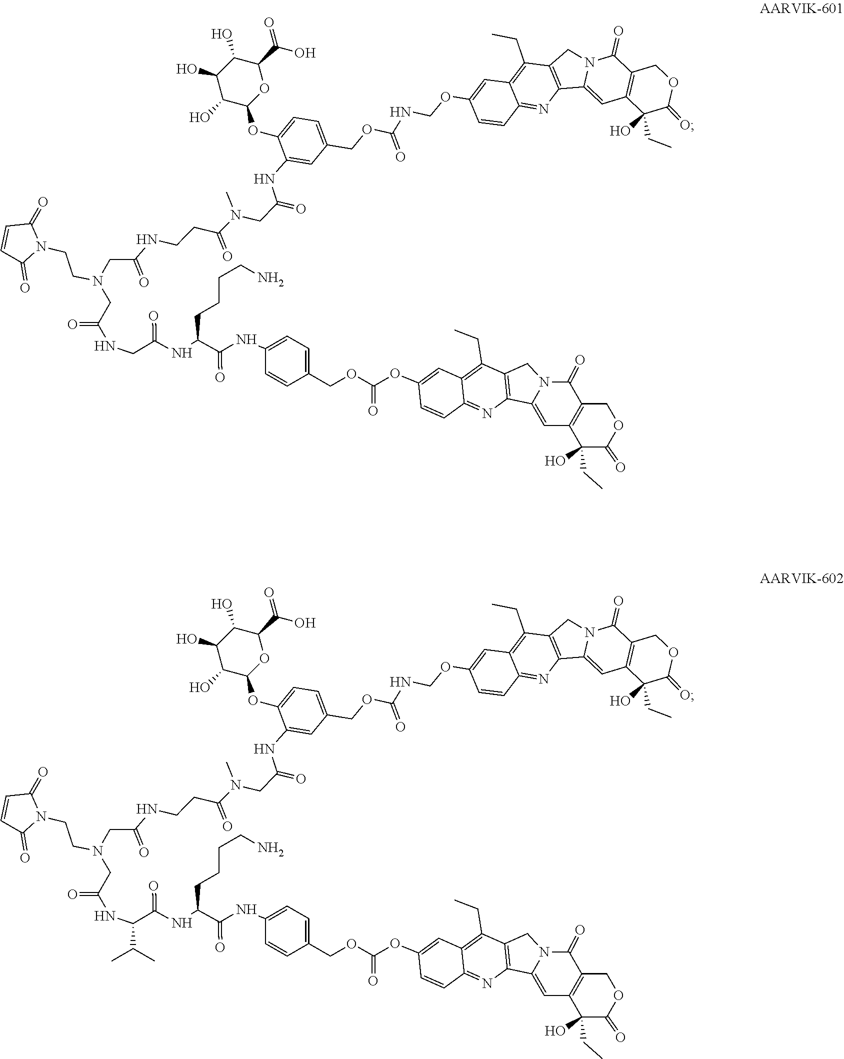

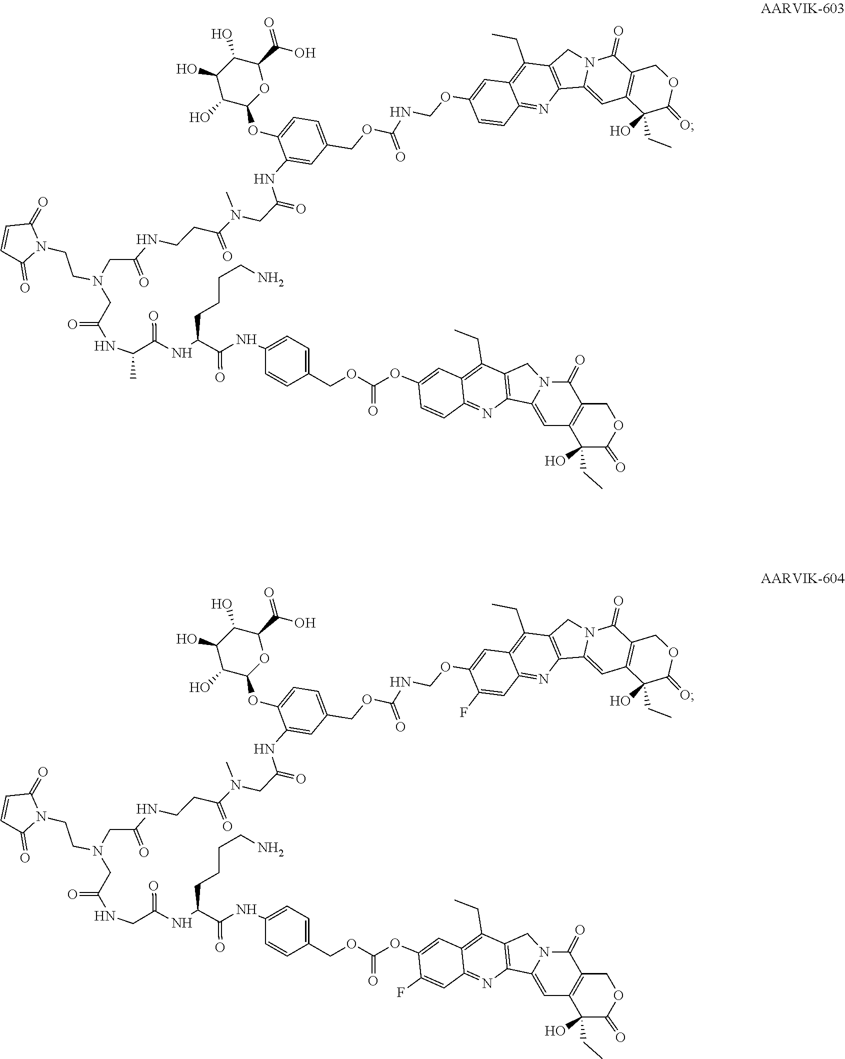

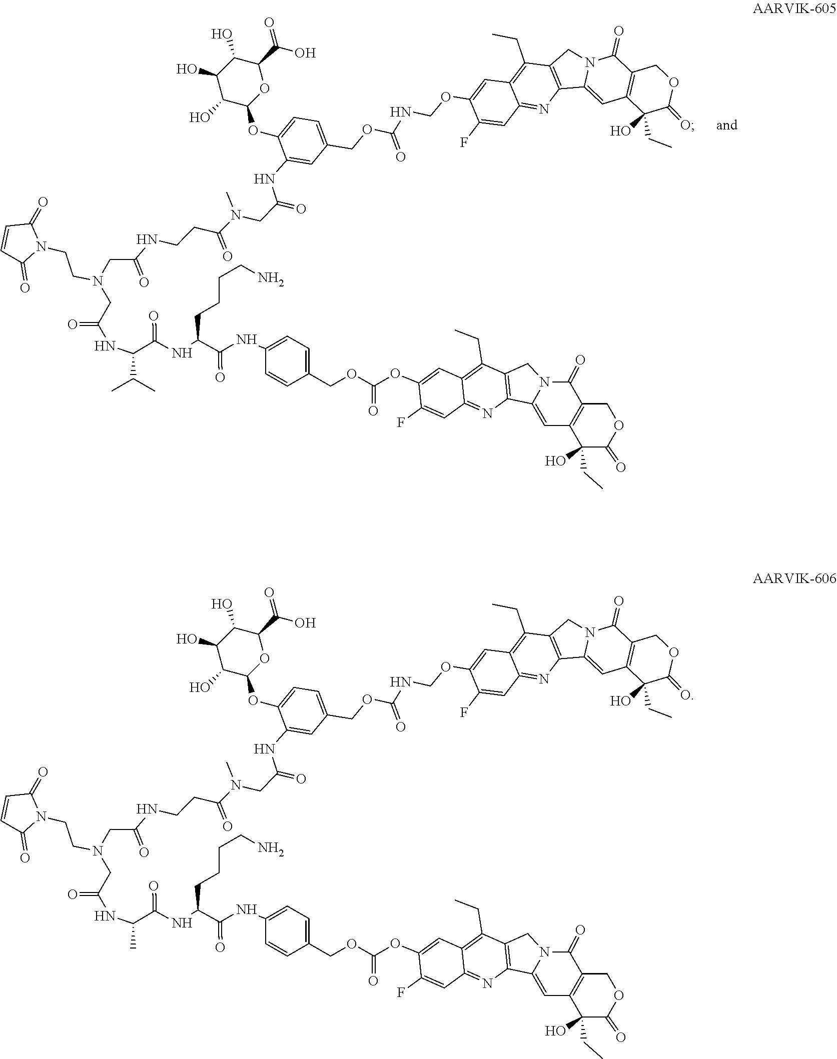

In another aspect provided herein is a compound of Formula (VI):

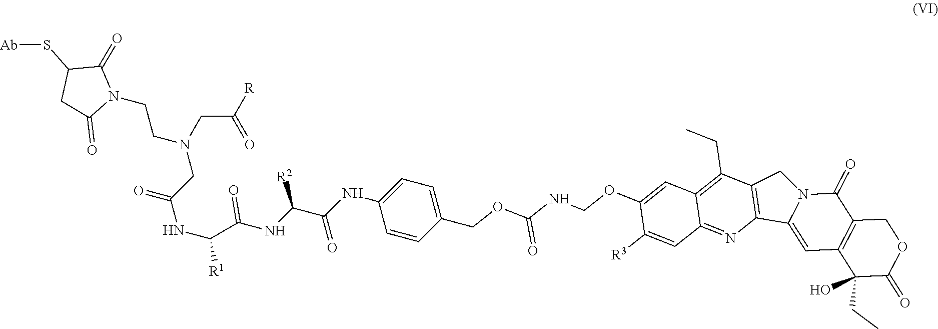

wherein Ab is an antibody; and

R is selected from the group consisting of Formula (II), Formula (III), and Formula (IV):

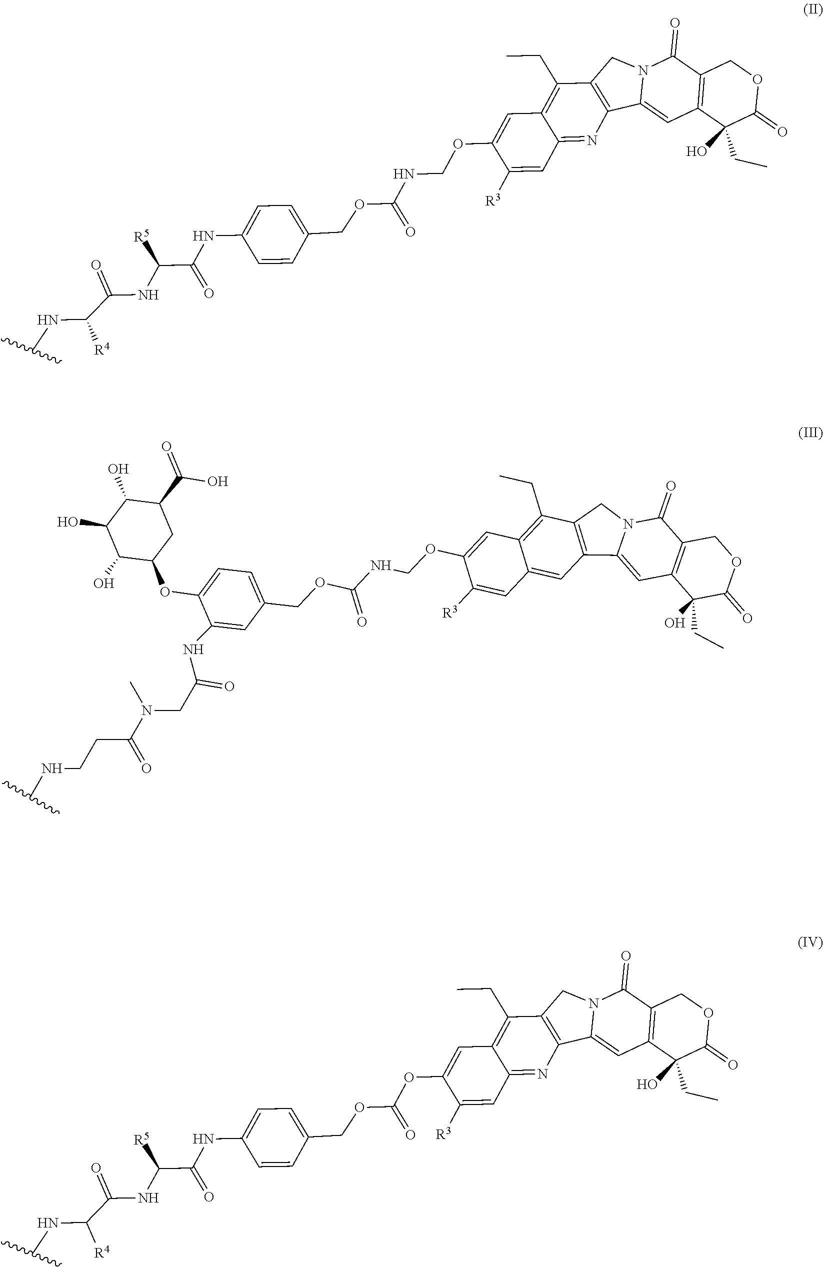

R¹ is —CH₃ or —CH(CH₃)₂;

R² is —CH₃, —(CH₂)₃NHC(═O)NH₂ or —CH(CH₃)₂;

R³ is independently selected from H and F (this is, both the same or different);

R⁴ is —H, —CH₃ or —CH(CH₃)₂;

R⁵ is —CH₃, —(CH₂)₄NH₂, —(CH₂)₃NHC(═O)NH₂ or —CH(CH₃)₂;

or a pharmaceutically acceptable salt thereof.

In another aspect provided herein is a compound of Formula (VII)

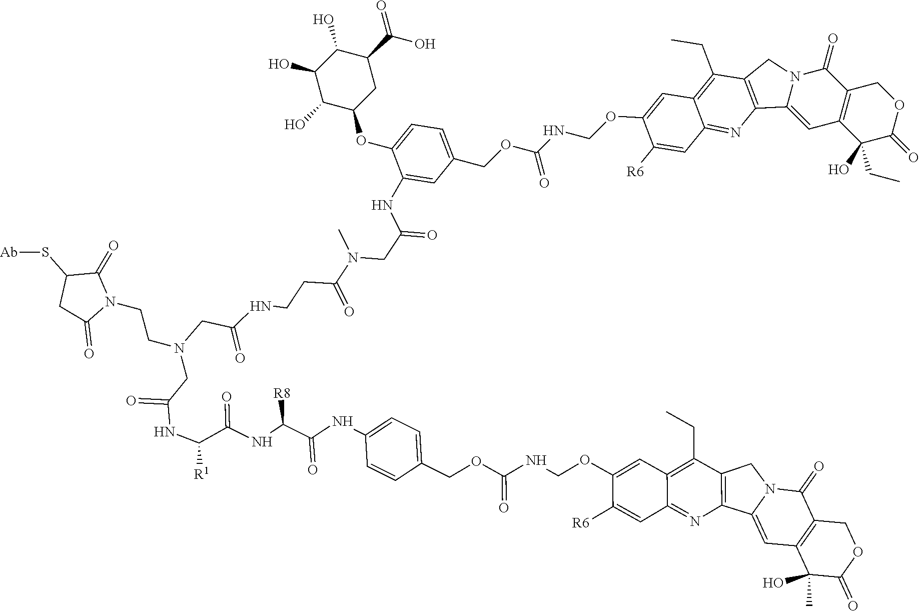

wherein Ab is an antibody; and

R⁶ is H or F;

R⁷ is —H, —CH₃ or —CH(CH₃)₂;

R⁸ is —CH₃, —(CH₂)₄NH₂, —(CH₂)₃NHC(═O)NH₂ or —CH(CH₃)₂;

or a pharmaceutically acceptable salt thereof.

In one embodiment, the antibody comprises a whole antibody. In another embodiment, the antibody comprises an antibody fragment. In another embodiment, the antibody fragment comprises a Fab, F(ab′)2, Fv, scFv, sc(Fv)2, bispecific scFv, bispecific sc(Fv)2, minibody, diabody, triabody, nanobody, or Fd. In another embodiment, the antibody comprises an IgA, IgD, IgE, IgG, or IgM. In another embodiment, the antibody is a monoclonal antibody. In another embodiment, the antibody is a humanized antibody. In another embodiment, the antibody is further conjugated to a toxin, a drug, a radioisotope, or a chemotherapeutic agent. In another embodiment, the antibody comprises a dissociation constant higher than the nanomole range. In another embodiment, the antibody comprises a dissociation constant higher than the picomole range. In another embodiment, the antibody comprises a dissociation constant higher than the femtomole range.

In another embodiment, the carrier comprises a buffer. In another embodiment, the buffer comprises normal saline, phosphate-buffered saline, Hank's balanced salt solution, or PlasmaLyte ATM. In another embodiment, the carrier comprises an antioxidant. In another embodiment, the antioxidant comprises ascorbic acid, glutathione, cysteine, methionine, citric acid, or a combination thereof. In another embodiment, the carrier comprises a preservative. In another embodiment, the preservative comprises ethanol, benzyl alcohol, phenol, m-cresol, p-chlor-m-cresol, methyl or propyl parabens, benzalkonium chloride, or a combination thereof. In another embodiment, the carrier comprises an amino acid. In another embodiment, the amino acid comprises arginine, glycine, ornithine, lysine, histidine, glutamic acid, aspartic acid, isoleucine, leucine, alanine, phenylalanine, tyrosine, tryptophan, methionine, serine, proline, or a combination thereof. In another embodiment, the carrier comprises a monosaccharide or a disaccharide. In another embodiment, the carrier comprises a low molecular weight polypeptide. In another embodiment, the carrier comprises a protein. In another embodiment, the protein comprises gelatin and/or serum albumin. In another embodiment, the carrier comprises a chelating agent. In another embodiment, the chelating agent comprises EDTA and/or EGTA. In another embodiment, the carrier comprises a sugar. In another embodiment, the sugar comprises trehalose, sucrose, lactose, glucose, mannose, maltose, galactose, fructose, sorbose, raffinose, glucosamine, N-methylglucosamine, galactosamine, neuraminic acid, or a combination thereof. In another embodiment, the carrier comprises a non-ionic surfactant. In another embodiment, the non-ionic surfactant comprises Tween, Pluronics, Triton-X, polyethylene glycol, or a combination thereof.

In another aspect provided herein is a pharmaceutical composition comprising a therapeutically effective amount of the compound or salt of the compounds disclosed herein and a pharmaceutically acceptable carrier.

In another aspect provided herein is a kit comprising: a) a pharmaceutical composition as described herein; and b) instructions for use.

In another aspect provided herein is a method for the treatment of cancer, comprising administering a pharmaceutically acceptable amount of the pharmaceutical composition as provided herein. In one embodiment, the cancer compromises carcinomas of the bladder, breast, cervix, colon, endometrium, kidney, lung, esophagus, ovary, prostate, pancreas, skin, stomach, testes, or a blood born cancer (e.g., leukemia and/or lymphoma). In another embodiment, the subject comprises a mammal, e.g., a human, a non-human primate, a dog, cat, horse, bovine, rabbit, rat, mouse, goat, or pig. In another embodiment, the method further comprises administering an effective amount of a second therapeutic agent. In another embodiment, the second therapeutic agent comprises a second immunotherapy.

In another aspect, provided herein is a method comprising combining a compound as provided herein, and an antibody and providing conditions such that the compound conjugates to produce a drug-antibody conjugate.

In another aspect, provided herein is a method comprising contacting a cell with a compound or conjugate as provided herein. In one embodiment, the compound is internalized into the cell. In another embodiment, the compound is released from the antibody after internalization.

In another aspect, provided herein is a composition comprising a cell and a compound or conjugate as provided herein. In one embodiment, the compound is bound to a surface of the cell. In another embodiment, the antibody of the compound is bound to an antigen present on the surface of the cell. In another embodiment, the compound is within the cell. In another embodiment, the compound's drug and antibody are separate.

In another aspect provided herein is use of a compound as provided herein to produce an antibody-drug conjugate.

In another aspect provided herein is use of a compound as provided herein in the manufacture of a medicament.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art. The invention will be more particularly described in conjunction with the following drawings wherein:

FIG. 1 shows a scheme for the synthesis of Compound AARVIK-301.

FIG. 2 shows a scheme for the synthesis of Compound AARVIK-401.

FIGS. 3A and 3B together show a scheme for the synthesis of Compound AARVIK-501.

FIG. 4 shows a scheme for the synthesis of Compound AARVIK-601.

### DETAILED DESCRIPTION

This application is not limited to particular methodologies or the specific compositions described, as such may vary. It is also to be understood that the terminology used herein is for the purpose of describing particular embodiments only, and is not intended to be limiting, because the scope of the present application will be limited only by the appended claims and their equivalents.

Provided herein are novel double payload antibody-drug conjugates (ADCs), and payload-linker compounds, alternatively known as toxin-linker compounds, useful in connection with ADCs. The present invention further relates to pharmaceutical compositions including these ADCs and methods for using the compositions to treat cancer.

### I. Definitions and Abbreviations

Unless defined otherwise, all technical and scientific terms used herein have the same meaning as commonly understood by one of ordinary skill in the art to which this invention belongs. Although any methods and materials similar or equivalent to those described herein can be used in the practice or testing of the present application, the preferred methods and materials are now described.

As used herein, the term “epitope” refers to the localized site on an antigen that is recognized and bound by an antibody. Epitopes can include a few amino acids or portions of a few amino acids, e.g., 5 or 6, or more, e.g., 20 or more amino acids, or portions of those amino acids. In some cases, the epitope includes non-protein components, e.g., from a carbohydrate, nucleic acid, or lipid. In some cases, the epitope is a three-dimensional moiety. Thus, for example, where the target is a protein, the epitope can be comprised of consecutive amino acids, or amino acids from different parts of the protein that are brought into proximity by protein folding (e.g., a discontinuous epitope).

As used herein, the term “antibody” refers to a polypeptide comprising a framework region from an immunoglobulin gene that recognizes and specifically binds to a one or more target antigen(s), such as a protein, polypeptide, peptide, carbohydrate, polynucleotide, lipid or combinations thereof. This binding occurs through at least one antigen recognition site within the variable region of the immunoglobulin at one or more epitopes on the antigen. The variable region is most critical in binding specificity and affinity. As used herein, the term “antibody” encompasses intact polyclonal antibodies, intact monoclonal antibodies, antibody fragments, single chain Fv (scFv) mutants, multispecific antibodies, chimeric antibodies, humanized antibodies, human antibodies, hybrid antibodies, fusion proteins and any other immunoglobulin molecule comprising an antigen recognition site so long as the antibody exhibit the desired biological activity. Antibodies can be of (i) any of the five major classes of immunoglobulins, based on the identity of their heavy-chain constant domains—alpha (IgA), delta (IgD), epsilon (IgE), gamma (IgG) and mu (IgM), or (ii) subclasses (isotypes) thereof (E.g., IgG1, IgG2, IgG3, IgG4, IgA1 and IgA2). The light chains can be either lambda or kappa. Antibodies can be naked or conjugated to other molecules such as toxins, drugs, radioisotopes, chemotherapeutic agents, etc.

In one embodiment, an “intact antibody” comprises a tetramer composed of two identical pairs of polypeptide chains, each pair having one “light” (about 25 kD) and one “heavy” chain (about 50-70 kD). The heavy chain and light chains are connected through covalent and non-covalent bonds (e.g., disulfide linkage) that vary in number and amount between the various immunoglobulin classes. In one aspect, each chain comprises a variable region and a constant region. The antigen recognition site of the variable region is composed of hypervariable regions or complementarity determining regions (CDRs) and frameworks regions. The framework regions typically do not come into contact with the antigen but provide structural support for the CDRs. The constant region interacts with other immune cells of the body. Between the constant and variable region (IgG, IgD, IgA only but not IgM or IgE) is the hinge region in the center between the two heavy chains that provides flexibility to articulate antigen binding.

The following are a non-exhaustive list of different antibody forms, all retaining antigen binding activity:

(1) whole immunoglobulins (also referred to as “intact” antibodies) (two light chains and two heavy chains, e.g., a tetramer).

(2) an immunoglobulin polypeptide (a light chain or a heavy chain).

(3) an antibody fragment, such as Fv (a monovalent or bi-valent variable region fragment, and can encompass only the variable regions (e.g., VL and/or VH), Fab (VLCL VHCH), F(ab′)2, Fv (VLVH), scFv (single chain Fv) (a polypeptide comprising a VL and VH joined by a linker, e.g., a peptide linker), (scFv)2, sc(Fv)2, bispecific sc(Fv)2, bispecific (scFv)2, minibody (sc(FV)2 fused to CH3 domain), diabody (noncovalent dimer of single-chain Fv (scFv) fragment that consists of the heavy chain variable (VH) and light chain variable (VL) regions connected by a small peptide linker), triabody is trivalent sc(Fv)₃ or trispecific sc(Fv)₃.

(4) a multivalent antibody (an antibody comprising binding regions that bind two different epitopes or proteins, e.g., “scorpion” antibody.

(5) a fusion protein comprising a binding portion of an immunoglobulin fused to another amino acid sequence (such as a fluorescent protein).

As used herein, the term “antibody fragment” refers to a part or portion of an antibody or antibody chain comprising fewer amino acid residues than an intact or complete antibody or antibody chain and which binds the antigen or competes with intact antibody. Fragments can be obtained via chemical or enzymatic treatment of an intact or complete antibody or antibody chain. Fragments can also be obtained by recombinant means. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be constructed by recombinant expression techniques.

While various antibody fragments are defined in terms of products of the digestion of an intact antibody, one of skill will appreciate that such fragments may also be synthesized de novo chemically or constructed and expressed using recombinant DNA methodology.

A single chain Fv (scFv) refers to a polypeptide comprising a VL and VH joined by a linker, e.g., a peptide linker. ScFvs can also be used to form tandem (or di-valent) scFvs or diabodies. Production and properties of tandem scFvs and diabodies are described, e.g., in Asano et al. (2011) J Biol. Chem. 286:1812; Kenanova et al. (2010) Prot Eng Design Sel 23:789; Asano et al. (2008) Prot Eng Design Sel 21:597.

Antibody fragments further include Fd (the portion of the heavy chain included in the Fab fragment) and single domain antibodies. A single domain antibody (sdAb) is a variable domain of either a heavy chain or a light chain, produced by recombinant methods.

The phrase “CDR sequence set” as used herein refers to the 3 heavy chain and/or 3 light chain CDRs of a particular antibody described herein. A “light chain” CDR sequence set refers to the light chain CDR sequences. A “heavy chain” CDR sequence set refers to the heavy chain CDR sequences. A “full” CDR sequence set refers to both heavy chain and light chain CDR sequences. CDRs are predicted based on IMGT sequence alignment.

Knobs-into-holes (KIHs) technology involves engineering CH3 domains to create either a “knob” or a “hole” in each heavy chain to promote heterodimerization. In this approach a ‘knob’ variant can be obtained by replacement of a small amino acid with a larger one in the CH3 domain of an antibody (e.g., T366Y). The knob is designed to insert into a ‘hole’ in the CH3 domain of another antibody created by replacement of a large residue with a smaller one (e.g., Y407T).

As used herein, the term “monoclonal antibody” refers to a clonal preparation or composition of antibodies with a single binding specificity and affinity for a given epitope on an antigen (“monoclonal antibody composition”). A “polyclonal antibody” refers to a preparation or composition of antibodies that are raised against a single antigen, but with different binding specificities and affinities (“polyclonal antibody composition”).

As used herein, the term “chimeric antibody” refers to an antibody having amino acid sequences derived from two or more species. In one embodiment, the variable region of both light and heavy chains corresponds to the variable region of antibodies derived from one species of mammal (e.g., mouse, rat, rabbit, etc.) with the desired specificity, affinity and capability, while the constant regions are homologous to the sequence derived from another species (typically in the subject receiving the therapy, e.g., human) to avoid eliciting an immune response.

As used herein, the term “humanized antibody” refers to a chimeric antibody in which the CDRs, obtained from the VH and VL regions of a non-human antibody having the desired specificity, affinity and capability, are grafted to a human framework sequence. In one embodiment, the framework residues of the humanized antibody are modified to refine and optimize the antibody specificity, affinity and capability. Humanization, i.e., substitution of non-human CDR sequences for the corresponding sequences of a human antibody, can be performed following the methods described in, e.g., U.S. Pat. Nos. 5,545,806; 5,569,825; 5,633,425; 5,661,016; Riechmann et al., Nature 332:323-327 (1988); Marks et al., Bio/Technology 10:779-783 (1992); Morrison, Nature 368:812-13 (1994); Fishwild et al., Nature Biotechnology 14:845-51 (1996).

As used herein, the term “human antibody” refers to an antibody produced by a human or an antibody having an amino acid sequence corresponding thereto made by any technique known in the art.

As used herein, the term “hybrid antibody” refers to antibody in which pairs of heavy and light chains form antibodies with different antigenic determinant regions are assembled together so that two different epitopes or two different antigens can be recognized and bound by the resulting tetramer. Hybrid antibodies can be bispecific (binding 2 distinct antigens or epitopes) or multispecific (>1 distinct antigen or epitope).

As used herein, an antibody is “monospecific” if all of its antigen binding sites bind to the same epitope.

As used herein, an antibody is “multi-specific” if it has at least two different antigen binding sites which each bind to a different epitope or antigen. A bi-specific antibody binds to two different epitopes or antigens. A tri-specific antibody binds to three different epitopes or antigens. A tetra-specific antibody binds to four different epitopes or antigens.

As used herein, an antibody is “polyvalent” if it has more than one antigen binding site. For example, an antibody that is tetravalent has four antigen binding sites.

The specificity of the binding can be defined in terms of the comparative dissociation constants (Kd) of the antibody (or other targeting moiety) for target, as compared to the dissociation constant with respect to the antibody and other materials in the environment or unrelated molecules in general. A larger (higher) Kd is a Kd that describes a lower affinity interaction. Conversely a smaller (lower) Kd is a Kd that describes a higher affinity interaction or tighter binding. By way of example only, the Kd for an antibody specifically binding to a target may be femtomolar, picomolar, nanomolar, or micromolar and the Kd for the antibody binding to unrelated material may be millimolar or higher. Binding affinity can be in the micromolar range (Kd=10⁻⁴ to 10⁻⁶), nanomole range (Kd=10⁻⁷ M to 10⁻⁹ M), picomole range (Kd=10⁻¹⁰ M to 10⁻¹² M), or femtomole range (Kd=10⁻¹³ M to 10⁻¹⁵ M).

As used herein, an antibody “binds” or “recognizes” an antigen or epitope if it binds the antigen or epitope with a Kd of less than 10⁻⁴ M (i.e., in the micromolar range). The term “binds” with respect to a cell type (e.g., an antibody that binds cancer cells), typically indicates that an agent binds a majority of the cells in a pure population of those cells. For example, an antibody that binds a given cell type typically binds to at least ⅔ of the cells in a population of the indicated cells (e.g., 67, 75, 80, 85, 90, 91, 92, 93, 94, 95, 96, 97, 98, 99, or 100%). In some cases, binding to a polypeptide can be assayed by comparing binding of the antibody to a cell that presents the polypeptide to binding (or lack thereof) of the antibody to a cell that does not express the polypeptide. One of skill will recognize that some variability will arise depending on the method and/or threshold of determining binding. Affinity of an antibody for a target can be determined according to methods known in the art, e.g., as reviewed in Ernst et al. Determination of Equilibrium Dissociation Constants, Therapeutic Monoclonal Antibodies (Wiley & Sons ed. 2009).

As used herein, the term “greater affinity” as used herein refers to a relative degree of antibody binding where an antibody X binds to target Y more strongly (Kon) and/or with a smaller dissociation constant (Koff) than to target Z, and in this context antibody X has a greater affinity for target Y than for Z. Likewise, the term “lesser affinity” herein refers to a degree of antibody binding where an antibody X binds to target Y less strongly and/or with a larger dissociation constant than to target Z, and in this context antibody X has a lesser affinity for target Y than for Z. The affinity of binding between an antibody and its target antigen, can be expressed as KA equal to 1/Kd where Kd is equal to kon/koff. The kon and koff values can be measured using surface plasmon resonance technology, for example, using a Molecular Affinity Screening System (MASS-1) (Sierra Sensors GmbH, Hamburg, Germany). An antagonist or blocking antibody is an antibody that partially or fully blocks inhibits or neutralizes a biological activity related to the target antigen relative to the activity under similar physiological conditions when the antibody is not present. Antagonists can be competitive, non-competitive or irreversible. A competitive antagonist is a substance that binds to a natural ligand or receptor at the same site as the natural ligand-receptor interaction or binds allosterically in a manner that induces a change to prevent normal binding. A non-competitive antagonist binds at a different site than the natural ligand-receptor interaction, but lower the Kd or signal resulting from the interaction. An irreversible inhibitor causes covalent modifications to the receptor preventing any subsequent binding.

As used herein, the term “avidity” refers to the overall stability of the binding complex between the antibody and the target antigen. It is governed by three factors, (i) the intrinsic affinity of the antibody for the antigen, (2) the valency of the antibody, and (3) the geometric arrangement of the interacting components. Affinity is the strength of the interaction between the antibody and a single target, whereas avidity is an accumulated strength of multiple affinities. In one embodiment, the antibodies provided herein are divalent.

As used herein, an antibody “preferentially binds” binds a first antigen relative to a second antigen if it binds the first antigen with greater affinity than it does the second antigen. Preferential binding can be at least any of 2-fold, 5-fold, 9-fold, 10-fold, 20-fold, 30-fold, 40-fold, 50-fold, 100-fold, 500-fold or 1000-fold greater affinity.

As used herein, an antibody “specifically binds” or is “specific for” a target antigen or target group of antigens if it binds the target antigen or each member of the target group of antigens with an affinity of at least any of 1×10⁻⁶ M, 1×10⁻⁷ M, 1×10⁻⁸ M, 1×10⁻⁹ M, 1× 10⁻¹⁰ M, 1×10⁻¹¹ M, 1×10⁻¹² M, and, for example, binds to the target antigen or each member of the target group of antigens with an affinity that is at least two-fold greater than its affinity for non-target antigens to which it is being compared. Typically, specific binding is characterized by binding the antigen with sufficient affinity that the antibody is useful as a diagnostic to detect the antigen or epitope and/or as a therapeutic agent in targeting the antigen or epitope.

As used herein, and antibody “blocks” or “antagonizes” the binding of a ligand to receptor when it competitively reduces or prevents interaction all of the ligand with the receptor. In an embodiment, the measured level of reduction can be at least any of 5%, 10%, 25%, 50%, 80%, 90%, 95%, 97.5%, 99%, 99.5%, 99.9% of a control (e.g., untreated) cell.

The term “captures” with respect to an antibody target (e.g., antigen, analyte, immune complex), typically indicates that an antibody binds a majority of the antibody targets in a pure population (assuming appropriate molar ratios). For example, an antibody that binds a given antibody target typically binds to at least ⅔ of the antibody targets in a solution (e.g., at least any of 67, 75, 80, 85, 90, 91, 92, 93, 94, 95, 96, 97, 98, 99, or 100%). One of skill will recognize that some variability will arise depending on the method and/or threshold of determining binding.

The term “cysteine-substituted antibody,” as used herein, refers to an antibody comprising at least one constant region immunoglobulin amino acid residue that has been substituted with a non-naturally occurring cysteine. A non-naturally occurring substitution is one that is not isotypic. In one embodiment, the substituted residues are heavy chain constant regions residues T1530, S1560, V266C, H285C, R3010, V303C, T307C, G3160, Y436C and L4410. In some embodiments, the constant region is of isotype IgG1, IgG2, IgG3 or IgG4.

The term “conjugate” refers to a first molecule, e.g., an antibody (an “immunoconjugate”), chemically coupled with a chemical moiety, such as a detectable label or a biologically active moiety, such as a drug, toxin or chemotherapeutic or cytotoxic agent or immune stimulator. Accordingly, this disclosure contemplates antibodies conjugated with one or more moieties. Furthermore, an antibody can be “conjugated antibody” or a “non-conjugated antibody” (that is, not conjugated with a moiety.

As used herein, the term “antibody-drug conjugate” or (“ADC”) refers to an antibody conjugated with a drug. Typically, conjugation involves covalent binding through a linker.

As used herein, the term “labeled” molecule (e.g., nucleic acid, protein, or antibody) refers to a molecule that is bound to a detectable label, either covalently, through a linker or a chemical bond, or noncovalently, through ionic, van der Waals, electrostatic, or hydrogen bonds, such that the presence of the molecule may be detected by detecting the presence of the detectable label bound to the molecule.

As used herein, the term “detectable label” refers to a composition detectable by spectroscopic, photochemical, biochemical, immunochemical, chemical, or other physical means. Examples of detectable labels are described herein and include, without limitation, colorimetric, fluorescent, chemiluminescent, enzymatic, and radioactive labels. For the purposes of the present disclosure, a detectable label can also be a moiety that does not itself produce a signal (e.g., biotin), but that binds to a second moiety that is able to produce a signal (e.g., labeled avidin).

The term “small molecule” refers to an organic or inorganic molecule having a size up to about 5000 Da, up to about 2000 Da, or up to about 1000 Da.

The term “cross-linked” with respect to an antibody refers to attachment of the antibody to a solid or semisolid matrix (e.g., Sepharose, beads, microtiter plate), or to another protein or antibody. For example, an antibody can be multimerized to create an antibody complex with multiple (more than 2) antigen-binding sites. The antibody can be multimerized by expressing the antibody as a high-valency isotype (e.g., IgA or IgM, which typically form complexes of 2 or 5 antibodies, respectively). Antibody multimerization can also be carried out by using a cross-linker comprising a reactive group capable of linking proteins (e.g., carbodiimide, NHS esters, etc.). Methods and compositions for cross-linking an antibody to a matrix are described, e.g., in the Abcam and New England Biolab catalogs and websites (available at abcam.com and neb.com). Cross-linker compounds with various reactive groups are described, e.g., in Thermo Fisher Scientific catalog and website (available at piercenet.com).

The term “substantial amount” refers to a majority, i.e., greater than 50% of a population, of a mixture or a sample.

The term “cytotoxic activity” or “cytotoxicity” refers to a cell-killing, a cytostatic or an anti-proliferative effect of an ADC or an intracellular metabolite of said ADC. Cytotoxic activity may be expressed as the IC₅₀ value, which is the concentration (molar or mass) per unit volume at which half the cells survive.

As used herein, the term terms “therapy,” “treatment,” “therapeutic intervention” and “amelioration” refer to any activity resulting in a reduction in the severity of symptoms. In the case of cancer, treatment can refer to, e.g., reducing tumor size, number of cancer cells, growth rate, metastatic activity, reducing cell death of non-cancer cells, reduced nausea and other chemotherapy or radiotherapy side effects, etc. The terms “treat” and “prevent” are not intended to be absolute terms. Treatment and prevention can refer to any delay in onset, amelioration of symptoms, improvement in patient survival, increase in survival time or rate, etc. Treatment and prevention can be complete (undetectable levels of neoplastic cells) or partial, such that fewer neoplastic cells are found in a patient than would have occurred without the present intervention. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects, the severity of disease is reduced by at least 25%, 50%, 75%, 80%, or 90%, or in some cases, no longer detectable using standard diagnostic techniques.

As used herein, the terms “effective amount,” “effective dose,” and “therapeutically effective amount,” refer to an amount of an agent, such as an antibody or immunoconjugate, that is sufficient to generate a desired response, such as reduce or eliminate a sign or symptom of a condition or ameliorate a disorder. In some examples, an “effective amount” is one that treats (including prophylaxis) one or more symptoms and/or underlying causes of any of a disorder or disease and/or prevents progression of a disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control.

As used herein, the term “pharmaceutical composition” refers to a composition comprising a pharmaceutical compound (e.g., a drug) and a pharmaceutically acceptable carrier.

As used herein, the term “pharmaceutically acceptable” refers to a carrier that is compatible with the other ingredients of a pharmaceutical composition and can be safely administered to a subject. The term is used synonymously with “physiologically acceptable” and “pharmacologically acceptable”. Pharmaceutical compositions and techniques for their preparation and use are known to those of skill in the art in light of the present disclosure. For a detailed listing of suitable pharmacological compositions and techniques for their administration one may refer to texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

The term “carrier” refers to a diluent, adjuvant or excipient, with which an ADC compound is administered. Such pharmaceutical carriers can be liquids, such as water and oils, including those of petroleum, animal, vegetable or synthetic origin. The carriers can be saline, and the like. In addition, auxiliary, stabilizing and other agents can be used. In one embodiment, when administered to a subject, the compound or conjugate and pharmaceutically acceptable carriers are sterile. Water is an exemplary carrier when the compound or conjugate are administered intravenously. Saline solutions and aqueous dextrose and glycerol solutions can also be employed as liquid carriers, particularly for injectable solutions. The present compositions, if desired, can also contain minor amounts of wetting or emulsifying agents, or pH buffering agents.

Pharmaceutically acceptable carriers will generally be sterile, at least for human use. A pharmaceutical composition will generally comprise agents for buffering and preservation in storage, and can include buffers and carriers for appropriate delivery, depending on the route of administration. Examples of pharmaceutically acceptable carriers include, without limitation, normal (0.9%) saline, phosphate-buffered saline (PBS) Hank's balanced salt solution (HBSS) and multiple electrolyte solutions such as PlasmaLyte ATM (Baxter).

Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA and/or EGTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Pluronics, Triton-X, or polyethylene glycol (PEG).

The terms “dose” and “dosage” are used interchangeably herein. A dose refers to the amount of active ingredient given to an individual at each administration. For the present invention, the dose can refer to the concentration of the antibody or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable label (if present). One of skill in the art will recognize that the dose can be modified depending on the above factors or based on therapeutic progress. The term “dosage form” refers to the particular format of the pharmaceutical and depends on the route of administration. For example, a dosage form can be in a liquid, e.g., a saline solution for injection.

As used herein, the term “subject” refers to an individual animal. The term “patient” as used herein refers to a subject under the care or supervision of a health care provider such as a doctor or nurse. Subjects include mammals, such as humans and non-human primates, such as monkeys, as well as dogs, cats, horses, bovines, rabbits, rats, mice, goats, pigs, and other mammalian species. Subjects can also include avians. A patient can be an individual that is seeking treatment, monitoring, adjustment or modification of an existing therapeutic regimen, etc. The term “cancer subject” refers to an individual that has been diagnosed with cancer. Cancer patients can include individuals that have not received treatment, are currently receiving treatment, have had surgery, and those that have discontinued treatment.

In the context of treating cancer, a subject in need of treatment can refer to an individual that has cancer or a pre-cancerous condition, has had cancer and is at risk of recurrence, is suspected of having cancer, is undergoing standard treatment for cancer, such as radiotherapy or chemotherapy, etc.

“Cancer”, “tumor,” “transformed” and like terms include precancerous, neoplastic, transformed, and cancerous cells, and can refer to a solid tumor, or a non-solid cancer (see, e.g., Edge et al. AJCC Cancer Staging Manual (7^{th} ed. 2009); Cibas and Ducatman Cytology: Diagnostic principles and clinical correlates (3^{rd} ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3^{rd} ed. 1994)). Although transformation can arise from infection with a transforming virus and incorporation of new genomic DNA, or uptake of exogenous DNA, it can also arise spontaneously or following exposure to a carcinogen.

The term “cancer” can refer to any cancer, including without limitation, leukemias, carcinomas, sarcomas, adenocarcinomas, lymphomas, solid and lymphoid cancers, etc. Examples of different types of cancer include, but are not limited to, lung cancer (e.g., non-small cell lung cancer or NSCLC), breast cancer, prostate cancer, colorectal cancer, bladder cancer, ovarian cancer, leukemia, liver cancer (i.e., hepatocarcinoma), renal cancer (i.e., renal cell carcinoma), thyroid cancer, pancreatic cancer, uterine cancer, cervical cancer, testicular cancer, esophageal cancer, stomach (gastric) cancer, kidney cancer, cancer of the central nervous system, skin cancer, glioblastoma and melanoma.

Unless otherwise indicated, the term “alkyl” by itself or as part of another term refers to a straight chain or branched, saturated hydrocarbon having the indicated number of carbon atoms (e.g., “C₁-C₈” alkyl refer to an alkyl group having from 1 to 8 carbon atoms). When the number of carbon atoms is not indicated, the alkyl group has from 1 to 8 carbon atoms, preferably from 1 to 6 carbon atoms. The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. Representative straight chain C₁-C₈ alkyls include, but are not limited to, methyl, ethyl, n-propyl, n-butyl, n-pentyl, n-hexyl, n-heptyl and n-octyl; while branched C₁-C₈ alkyls include, but are not limited to, -isopropyl, -sec-butyl, -isobutyl, -tert-butyl, -isopentyl, and -2-methylbutyl; unsaturated C₂-C₈alkyls include, but are not limited to, vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexyl, 2-hexyl, 3-hexyl, acetylenyl, propynyl, 1-butynyl, 2-butynyl, 1-pentynyl, 2-pentynyl and 3-methyl-1-butynyl.

Unless otherwise indicated, “alkylene,” by itself of as part of another term, refers to a saturated, branched or straight chain or cyclic hydrocarbon radical of the stated number of carbon atoms, typically 1-18 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethylene —CH₂CH₂—), 1,3-propylene (—CH₂CH₂CH₂—), 1,4-butylene (—CH₂CH₂CH₂CH₂—), and the like. A “C₁-C₁₀” straight chain alkylene is a straight chain, saturated hydrocarbon group of the formula —(CH₂)₁₋₁₀—. Examples of a C₁-C₁₀ alkylene include methylene, ethylene, propylene, butylene, pentylene, hexylene, heptylene, ocytylene, nonylene and decalene. In certain embodiments of the disclosure, alkylenes have from 1 to 9, from 1 to 8, from 1 to 7, and from 1 to 6 carbons.

The term “compound” or “compounds” as used herein refer to relates to novel double payload antibody-drug conjugates (ADCs), and payload-linker compounds, alternatively known as toxin-linker compounds, and includes any specific compounds encompassed by generic formulae disclosed herein. The compounds may be identified either by their chemical structure and/or chemical name. When the chemical structure and chemical name conflict, the chemical structure is determinative of the identity of the compound. The compounds may contain one or more chiral centers and/or double bonds and therefore, may exist as stereoisomers, such as double-bond isomers (i.e., geometric isomers), enantiomers or diastereomers. Accordingly, when the stereochemistry at chiral centers is not specified, the chemical structures depicted herein encompass all possible configurations at those chiral centers including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds also include isotopically labeled compounds where one or more atoms have an atomic mass different from the atomic mass conventionally found in nature. Examples of isotopes that may be incorporated into the compounds include, but are not limited to, ²H, ³H, ¹³C, ¹⁴C, ¹⁵N, ¹⁷O, and ¹⁸O. Compounds may exist in unsolvated forms as well as solvated forms, including hydrated forms and as N-oxides. In general, the hydrated, solvated and N-oxide forms are within the scope of the present disclosure. Certain compounds may exist in multiple crystalline or amorphous forms. In general, all physical forms are equivalent for the uses contemplated herein and are intended to be within the scope of the present disclosure. Further, it should be understood, when partial structures of the compounds are illustrated, that brackets indicate the point of attachment of the partial structure to the rest of the molecule.

Unless otherwise indicated, the term “heteroalkyl,” by itself or in combination with another term, means, unless otherwise stated, a stable straight or branched chain hydrocarbon, or combinations thereof, fully saturated or containing from 1 to 3 degrees of unsaturation, consisting of the stated number of carbon atoms and from one to three heteroatoms selected from the group consisting of O, N, Si, S and/or P, and wherein the nitrogen and sulfur atoms may optionally be oxidized and the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Up to two heteroatoms may be consecutive.

“Halo” or “halogen” refers to fluoro, chloro, bromo and iodo.

“Halo(C₁₋₆-alkyl)” refers to C₁₋₆-alkyl groups substituted with 1 to 3 or 1 to 2 halo groups, wherein C₁₋₆-alkyl and halo are as defined herein. The term includes, for example, CF₃.

The term “hydroxy” refers to the group —OH.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

The terms “racemic mixture” and “racemate” refer to an equimolar mixture of two enantiomeric species, devoid of optical activity.

An amino acid “derivative” includes an amino acid having substitutions or modifications by covalent attachment of a parent amino acid, such as, e.g., by alkylation, glycosylation, acetylation, phosphorylation, and the like. Further included within the definition of “derivative” is, for example, one or more analogs of an amino acid with substituted linkages, as well as other modifications known in the art.

A “natural amino acid” refers to arginine, glutamine, phenylalanine, tyrosine, tryptophan, lysine, glycine, alanine, histidine, serine, proline, glutamic acid, aspartic acid, threonine, cysteine, methionine, leucine, asparagine, isoleucine, and valine, unless otherwise indicated by context.

The phrase “pharmaceutically acceptable salt,” as used herein, refers to pharmaceutically acceptable organic or inorganic salts of a compound. The compound typically contains at least one amino group, and accordingly acid addition salts can be formed with this amino group. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

The terms “loading” or “drug loading” represent or refer to the average number of cytotoxic compounds per antibody in an ADC molecule. Drug loading may range from 1 to 10 drugs per antibody. This is sometimes referred to as the DAR, or drug to antibody ratio. Compositions of the ADCs described herein typically have DAR's of from 1-20, and in certain embodiments from 1-8, from 2-8, from 2-6, from 2-5 and from 2-4. Typical DAR values are 2, 4, 6 and 8. The average number of drugs per antibody, or DAR value, may be characterized by conventional means such as UV/visible spectroscopy, mass spectrometry, ELISA assay, and HPLC. The quantitative DAR value may also be determined. In some instances, separation, purification, and characterization of homogeneous ADCs having a particular DAR value may be achieved by means such as reverse phase HPLC or electrophoresis.

DAR may be limited by the number of attachment sites on the antibody. Two typical means of attachment are via a sulfide linkage with a cysteine thiol residue and an amide linkage with a lysine residue. See Puthenveetil, S., et al., Bioconjugate Chem., 2016, 27:1880-1888. For example, where the attachment is a cysteine thiol, an antibody may have only one or several cysteine thiol groups, or may have only one or several sufficiently reactive thiol groups through which a Linker unit may be attached. In some embodiments, the cysteine thiol is a thiol group of a cysteine residue that forms an interchain disulfide bond. In some embodiments, the cysteine thiol is a thiol group of a cysteine residue that does not form an interchain disulfide bond. Most cysteine thiol residues in the antibodies exist as disulfide bridges and must be reduced with a reducing agent such as dithiothreitol (DTT). Typically, fewer than the theoretical maximum of drug moieties are conjugated to an antibody during a conjugation reaction. An antibody may contain, for example, many lysine residues that do not react with a linker or linker intermediate. Only the most reactive lysine groups may react with a reactive linker reagent to form an amide bond with the antibody. The antibody may be subjected to denaturing conditions to reveal reactive nucleophilic groups such as lysine or cysteine.

The loading (drug/antibody ratio) of an ADC may be controlled in several different manners, including: (i) limiting the molar excess of drug-linker relative to the antibody, (ii) limiting the conjugation reaction time or temperature, and (iii) partial or limiting reductive conditions for cysteine thiol modification. Where more than one nucleophilic group reacts with a drug-linker then the resulting product is a mixture of ADCs with a distribution of one or more drugs moieties per antibody. The average number of drugs per antibody may be calculated from the mixture by, for example, dual ELISA antibody assay, specific for antibody and specific for the drug. Individual ADCs may be identified in the mixture by mass spectroscopy, and separated by HPLC, e.g., hydrophobic interaction chromatography.

The term “protecting group” refers to a grouping of atoms that when attached to a reactive functional group in a molecule masks, reduces or prevents reactivity of the functional group. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

Reference will now be made in detail to certain preferred methods of treatment, compounds and methods of administering these compounds. The invention is not limited to those preferred compounds and methods but rather is defined by the claim(s) issuing here from.

### II. Double Payload Linker Compounds and ADCs Thereof

Double payload ADCs with linkers cleavable at multiple locations are new. In the present disclosure, the ADCs are synthesized by forming a covalent bond from a maleimide moiety to two multiply cleavable linkers covalently bonded to a known cytotoxic payload. The payload is known and comprises the ethyl analog SN-38 of camptothecin, having the IUPAC name (4S)-4,11-Diethyl-4,9-dihydroxy-1,4-dihydro-3H,14H-pyrano[3′,4′:6,7]indolizino[1,2-b]quinoline-3,14-dione, also known as 7-ethyl-10-hydroxy-camptothecin. Camptothecin is an alkaloid isolated from the stem wood of the Chinese tree, Camptotheca acuminata. The compound selectively inhibits the nuclear enzyme DNA topoisomerase, type I, as does SN-28, demonstrating antitumor activity. The present disclosure utilizes SN-38 and its fluorine-substituted analogs as payloads.

ADCs with camptothecin and its analogs are also known (Li, W., et al., ACS Med. Chem. Lett., 2019, 10:1386-1392). However, these ADCs use conventional linkers to bind the camptothecin payload to an antibody, with only one payload per linker chain (WO 2019/236954; Scott, J., et al.) The present disclosure delivers two SN-38 payloads, or fluorine-containing analogs thereof, per covalent attachment via a maleimide moiety that binds to a cysteine residue on the antibody.

The linker from each payload to the maleimide moiety may be the same or different. Each double payload-linker-maleimide compound described herein is novel. The linkers are chosen for their ability to release the payloads via different mechanisms or combinations thereof.

These different release mechanisms are, for example, enzyme cleavable or acid-labile. Using different choice of lysosomal enzymes (cathepsin cleavable linkers or beta-glucuronidase cleavable linkers) improves the linker cleavage by lowering the burden on a particular class of lysosomal enzymes. In addition, combining acid-labile type linker, the linker cleavage can adopt different release conditions such as acidic pH in endosomes or acidic tumor microenvironment, thus by not limiting the linker cleavage only in the lysosomes. For example, Her2 antigen-antibody complex predominantly recycles back to cell surface via recycling endosomes and a minority fraction enters into lysosomal compartment. Having a dual linker with acid-labile and enzyme cleavable linker release mechanisms can efficiently release the payload both in endosomes as well as lysosomes in addition to releasing the payload in other acidic tumor microenvironments.

### III. Antibody Moiety

As noted above, the term “antibody” (or “Ab” or “AB”) herein is used in the broadest sense and specifically covers intact monoclonal antibodies, polyclonal antibodies, monospecific antibodies, multispecific antibodies (e.g., bispecific antibodies), and antibody fragments that exhibit the desired biological activity. In addition, while certain aspects of the disclosure described herein refer to antibody-drug conjugates, it is further envisioned that the antibody portion of the conjugate might be replaced with anything that specifically binds or reactively associates or complexes with a receptor, antigen or other receptive moiety associated with a given target-cell population. For example, instead of containing an antibody, a conjugate of the disclosure could contain a targeting molecule that binds to, complexes with, or reacts with a receptor, antigen or other receptive moiety of a cell population sought to be therapeutically or otherwise biologically modified. Example of such molecules include smaller molecular weight proteins, polypeptide or peptides, lectins, glycoproteins, non-peptides, vitamins, nutrient-transport molecules (such as, but not limited to, transferrin), or any other cell binding molecule or substances. In certain aspects, the antibody or other such targeting molecule acts to deliver a drug to the particular target cell population with which the antibody or other targeting molecule interacts.

The present invention may also comprise multivalent antibodies, which can enhance the repeat binding of antibody molecules to a given tumor antigen. Typically, multivalent antibodies will also enhance internalization due to antigen-antibody complex clustering on the cell surface. Both these factors can contribute to increasing the ADC delivery into the cells.

The distance between the payload(s) and the antibody is optimized in the present disclosure for delivery to the site of desired activity. Multiple double payload compounds are conjugated to each antibody and a loading factor for payload/antibody may be determined by the methods described below.

In another aspect, the present disclosure relates to an antibody-drug conjugate compound of Formula (VI) or Formula (VII) wherein the antibody Ab is selected from: gemtuzumab, trastuzumab (Herceptin®), pertuzumab (Perjeta®), iratumumab, inotuzumab, pinatuzumab, epratuzumab, polatuzumab, coltuximab, lovotuzumab, sacituzumab, anetumab, aprutumab, aratumumab, atezolizumab, avelumab, azintuxizumab, bevacizumab (Avastin®), bivatuzumab, brentuximab, camidanlumab, cantuzumab, cetuximab (Erbitux®), cofetuzumab, denintuzumab, durvalumab, elotuzumab, enfortumab, glembatumumab, ibritumomab, iladatuzumab, indatuximab, industuzumab, labetuzumab, ladiratuzumab, laprituximab, lifastuzumab, loncastuximab, lorvotuzumab, lupartumab, milatuzumab, mirvetuximab, naratuximab, natalizumab, necitumumab, obinutuzumab, ocrelizumab, ofatumumab, olaratumab, panitumumab, pertuzumab, rituximab (Rituxan®), rovalpituzumab, sirtratumab, sofituzumab, telisotuzumab, tositumomab, trastuzumab, and vadastuximab. In certain preferred embodiments, the antibody Ab is selected from the group consisting of: trastuzumab, pertuzumab, cetuximab, panitumumab, atezolizumab or pembrolizumab.

Heteroatoms that may be present on an antibody moiety include sulfur (in one embodiment, from a sulfhydryl group of an antibody), oxygen (in one embodiment, from a carbonyl, carboxyl or hydroxyl group of an antibody) and nitrogen (in one embodiment, from a primary or secondary amino group of an antibody). These hetero atoms can be present on the antibody in the antibody's natural state, for example a naturally-occurring antibody, or can be introduced into the antibody via chemical modification.

In one embodiment, an antibody moiety has a sulfhydryl group and the antibody moiety bonds via the sulfhydryl group's sulfur atom.

Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Useful monoclonal antibodies include, but are not limited to, human monoclonal antibodies, humanized monoclonal antibodies, antibody fragments, or chimeric monoclonal antibodies. Human monoclonal antibodies may be made by any of numerous techniques known in the art.

The antibody can also be a bispecific antibody. Methods for making bispecific antibodies are known in the art.

The antibody can be a functionally active fragment, derivative or analog of an antibody that immunospecifically binds to target cells (e.g., tumor-associated antigens, cancer cell antigens, viral antigens, or microbial antigens) or other antibodies that bind to tumor cells or matrix. In this regard, “functionally active” means that the fragment, derivative or analog is able to elicit anti-anti-idiotype antibodies that recognize the same antigen that the antibody from which the fragment, derivative or analog is derived recognized. Specifically, in an exemplary embodiment the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art (e.g., the BIA core assay).

Other useful antibodies include fragments of antibodies such as, but not limited to, F(ab′)₂ fragments, Fab fragments, Fvs, single chain antibodies, diabodies, triabodies, tetrabodies, scFv, scFv-FV, or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as for example, those having a variable region derived from a murine monoclonal and human immunoglobulin constant regions. Humanized antibodies are antibody molecules from non-human species having one or more complementarity determining regions (CDRs) from the non-human species and a framework region from a human immunoglobulin molecule. Such chimeric and humanized monoclonal antibodies can be produced by recombinant DNA techniques known in the art.

Completely human antibodies are particularly desirable and can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the disclosure. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies using methods known to one skilled in the art. Other human antibodies can be obtained commercially from numerous companies.

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. Human antibodies can also be produced using various techniques known in the art, including phage display libraries.

In other embodiments, the antibody is a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, preferably at least 10, 20 or 50 amino acid portion of the protein) that is not from an antibody. Preferably, the antibody or fragment thereof is covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, but not by way of limitation, derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody moiety or other protein, etc. Any of numerous chemical modifications can be carried out by known techniques including, but not limited to, specific chemical cleavage, acetylation, formylation, metabolic synthesis in the presence of tunicamycin, etc. Additionally, the analog or derivative can contain one or more unnatural amino acids.

Antibodies can have modifications (e.g., substitutions, deletions or additions) in amino acid residues that interact with Fc receptors. In particular, antibodies can have modifications in amino acid residues identified as involved in the interaction between the anti-Fc domain and the FcRn receptor.

Antibodies immunospecific for a cancer cell antigen can be obtained commercially or produced by any method known to one of skill in the art such as, e.g., chemical synthesis or recombinant expression techniques. The nucleotide sequence encoding antibodies immunospecific for a cancer cell antigen can be obtained, e.g., from the GenBank database or a database like it, literature publications, or by routine cloning and sequencing.

In certain embodiments, the present disclosure relates to any of the aforementioned antibody-drug conjugates and attendant definitions, wherein the antibody-drug conjugate comprises between 1, 2, 3, 4, 5, 6, 7, 8, 9 or 10 compounds of the disclosure. In certain exemplary embodiments, the antibody-drug conjugate comprises 1, 2, 3 or 4 compounds of the disclosure.

The number of payload molecules covalently linked to an antibody may vary according to the specific toxic compound and antibody. In one embodiment, 1 to 4 toxins are linked. In another embodiment from about 1 to about 4 toxins are linked. In a further embodiment, from about 1 to about 6 toxins are linked.

The loading (drug/antibody ratio) of an antibody-drug conjugate may be controlled in different ways, and for example, by: (i) limiting the molar excess of the drug-linker intermediate compound relative to antibody, (ii) limiting the conjugation reaction time or temperature, and (iii) partial or limiting reductive conditions for cysteine thiol modification. By “drug” is meant payload or cytotoxic compound.

The average number of drugs per antibody may be calculated from the mixture by a dual ELISA antibody assay, which is specific for antibody and specific for the drug. Individual antibody-drug conjugate molecules may be identified in the mixture by mass spectroscopy and separated by HPLC, e.g., hydrophobic interaction chromatography (see, e.g., McDonagh, et al., Prot. Engr. Design Sel., 2006, 19(7):299-307; Hamblett, et al., Clin. Cancer Res., 2004, 10:7063-7070; Hamblett, K. J., et al. “Effect of drug loading on the pharmacology, pharmacokinetics, and toxicity of an anti-CD30 antibody-drug conjugate,” Abstract No. 624, American Association for Cancer Research, 2004 Annual Meeting, Mar. 27-31, 2004, Proceedings of the AACR, Volume 45, March 2004; Alley, S. C., et al., “Controlling the location of drug attachment in antibody-drug conjugates,” Abstract No. 627, American Association for Cancer Research, 2004 Annual Meeting, Mar. 27-31, 2004, Proceedings of the AACR, Volume 45, March 2004). In certain embodiments, a homogeneous antibody-drug conjugate with a single loading value may be isolated from the conjugation mixture by electrophoresis or chromatography.

### IV. In Vitro Cell Proliferation Assays

The antibody-drug conjugates of the disclosure can be evaluated for their ability to suppress the proliferation of various cancer cell lines in vitro.

Generally, the cytotoxic or cytostatic activity of an ADC is measured by exposing mammalian cells having receptor proteins, e.g., HER2, to the antibody of the ADC in a cell culture medium, culturing the cells for a period from about 6 hours to about 5 days, and measuring cell viability. Cell-based in vitro assays are used to measure viability (proliferation), cytotoxicity, and induction of apoptosis (caspase activation) of the ADC of the disclosure. For example, the in vitro potency of an ADC is measured by a cell proliferation assay. The CellTiter-Glo® Luminescent Cell Viability Assay is a commercially available (Promega Corp., Madison, Wis.), homogeneous assay method based on the recombinant expression of Coleoptera luciferase (U.S. Pat. Nos. 5,583,024; 5,674,713; and 5,700,670). This cell proliferation assay determines the number of viable cells in culture based on quantitation of the ATP present, an indicator of metabolically active cells (Crouch, et al., J. Immunol. Meth., 1993, 160:81-88; U.S. Pat. No. 6,602,677). The CellTiter-Glo® Assay is conducted in 96-well format, making it amenable to automated high-throughput screening (HTS) (Gree, et al., AntiCancer Drugs, 1995, 6:398-404). The homogeneous assay procedure involves adding the single reagent (CellTiter-Glo® Reagent) directly to cells cultured in serum-supplemented medium. Cell washing, removal of medium and multiple pipetting steps are not required. The system detects as few as 15 cells/well in a 384-well format in 10 minutes after adding reagent and mixing. The cells may be treated continuously with the ADC, or they may be treated and separated from the ADC. Generally, cells treated briefly, i.e., 3 hours, showed the same potency effects as continuously treated cells.

### V. In Vivo Efficacy

The in vivo efficacy of an ADC of the disclosure can be tested by tumor xenograft studies in mice to measure target-dependent and dose-dependent potency in inhibition of tumor growth. Efficacy of the ADC may correlate with target antigen expression of the tumor cells.

The efficacy of the ADC is measured in vivo by implanting allografts or xenografts of cancer cells in rodents and treating the tumors with the ADC. Variable results are to be expected depending on the cell line, the dose of the cytotoxic drug, the specificity of antibody binding of the ADC to receptors present on the cancer cells, dosing regimen, and other factors. The in vivo efficacy of the cytotoxic drug or ADC can be measured using a transgenic explant mouse model expressing moderate to high levels of a tumor-associated antigen, including Her2-expressing KPL4, and CD22-expressing BJAB. Subjects may be treated once with the cytotoxic drug or ADC and monitored over several, e.g., 3-6, weeks to measure the time to tumor doubling, log cell kill, and tumor shrinkage. Follow up dose-response and multi-dose experiments may be conducted.

For example, the in vivo efficacy of an anti-HER2 ADC of the disclosure can be measured by a high expressing HER2 transgenic explant mouse model (Phillips, et al., Cancer Res., 2008, 68:9280-90). An allograft is propagated from the Fo5 mmtv transgenic mouse which does not respond to, or responds poorly to, HERCEPTIN® (Genentech, Inc.) therapy. Subjects are treated once or more with ADC at certain dose levels (mg/kg) and placebo buffer control (Vehicle) and monitored over two weeks or more to measure the time to tumor doubling, log cell kill, and tumor shrinkage.

### VI. Pharmaceutical Compositions and Methods of Administration

In other embodiments, another aspect of the disclosure relates to pharmaceutical compositions or dosage forms including an effective amount of the antibody-drug conjugate of the disclosure and a pharmaceutically acceptable carrier or vehicle.

The present pharmaceutical compositions can be in any form that allows for the composition to be administered to a subject. For example, the composition can be in the form of a solid or liquid. Typical routes of administration include, without limitation, parenteral, ocular and intra-tumor. Parenteral administration includes subcutaneous injections, intravenous, intramuscular or intrasternal injection or infusion techniques. In one aspect, the compositions are administered parenterally. In a specific embodiment, the compositions are administered intravenously.

Pharmaceutical compositions can be formulated so as to allow the ADC to be bioavailable upon administration of the composition to a subject. Compositions can take the form of one or more dosage units, where for example, a tablet can be a single dosage unit, and a container of a compound of the antibody-drug conjugate thereof in liquid form can hold a plurality of dosage units.

The pharmaceutically acceptable carrier or vehicle can be solid or particulate, so that the compositions are, for example, in tablet or powder form. The carrier(s) can be liquid. In addition, the carrier(s) can be particulate.

The composition can be in the form of a liquid, e.g., a solution, emulsion or suspension. In a composition for administration by injection, one or more of a surfactant, preservative, wetting agent, dispersing agent, suspending agent, buffer, stabilizer and isotonic agent can also be included.

The liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or diglycerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates, phosphates or amino acids and agents for the adjustment of tonicity such as sodium chloride or dextrose. A parenteral composition can be enclosed in ampoule, a disposable syringe or a multiple-dose vial made of glass, plastic or other material. Physiological saline is an exemplary adjuvant. An injectable composition is preferably sterile.

The amount of the antibody-drug conjugate that is effective in the treatment of a particular condition will depend on the nature of the condition and can be determined by standard clinical techniques. In addition, in vitro or in vivo assays can be employed to help identify optimal dosage ranges. The precise dose to be utilized in the compositions will also depend on the route of administration and should be decided according to the judgment of the practitioner and each subject's circumstances. The specific dose level for any particular individual will depend upon a variety of factors including the activity of the compound or drug-antibody conjugate, the age, body weight, general physical and mental health, genetic factors, environmental influences, sex, diet, time of administration, route of administration, rate of excretion, and the severity of the particular problem being treated.

The compositions comprise an effective amount of the ADC of the disclosure such that a suitable dosage will be obtained. Typically, this amount is at least about 0.01% of the ADC by weight of the composition. In an exemplary embodiment, pharmaceutical compositions are prepared so that a parenteral dosage unit contains from about 0.01% to about 2% by weight of the amount of an ADC of the disclosure.

For intravenous administration, the composition can comprise from about 0.01 to about 100 mg of an ADC of the disclosure per kg of the subject's body weight. In one aspect, the composition can include from about 1 to about 100 mg of an ADC of the disclosure per kg of the subject's body weight. In another aspect, the amount administered will be in the range from about 0.1 to about 25 mg/kg of body weight of the ADC.

Generally, the dosage of an ADC of the disclosure administered to a subject is typically about 0.01 mg/kg to about 20 mg/kg of the subject's body weight. In one aspect, the dosage administered to a subject is between about 0.01 mg/kg to about 10 mg/kg of the subject's body weight. In another aspect, the dosage administered to a subject is between about 0.1 mg/kg and about 10 mg/kg of the subject's body weight. In yet another aspect, the dosage administered to a subject is between about 0.1 mg/kg and about 5 mg/kg of the subject's body weight. In yet another aspect the dosage administered is between about 0.1 mg/kg to about 3 mg/kg of the subject's body weight. In a further aspect, the dosage administered is between about 1 mg/kg to about 3 mg/kg of the subject's body weight.

In specific embodiments, it can be desirable to administer one or more ADCs of the disclosure locally to the area in need of treatment. This can be achieved, for example, and not by way of limitation, by local infusion during surgery; topical application, e.g., in conjunction with a wound dressing after surgery; by injection; by means of a catheter; or by means of an implant, the implant being of a porous, non-porous, or gelatinous material, including membranes, such as sialastic membranes, or fibers. In one embodiment, administration can be by direct injection at the site (or former site) of a cancer, tumor or neoplastic or pre-neoplastic tissue.

In yet another embodiment, the ADC of the disclosure can be delivered in a controlled release system, such as but not limited to, a pump or various polymeric materials can be used. In yet another embodiment, a controlled-release system can be placed in proximity of the target of the ADC, e.g., the liver, thus requiring only a fraction of the systemic dose.

In an embodiment, the ADCs are formulated in accordance with procedures known to those skilled in the art for the manufacture of a pharmaceutical composition adapted for intravenous administration. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule indicating the quantity of active agent. Where an ADC of the disclosure is to be administered by infusion, it can be dispensed, for example, with an infusion bottle containing sterile pharmaceutical grade water or saline. Where the ADC is administered by injection, an ampoule of sterile water for injection or saline can be provided so that the ingredients can be mixed prior to administration.

Whether in solid or liquid form, the present compositions can include an additional therapeutic agent(s) used in the treatment of cancer. For example, the antibody-drug conjugates can be administered in combination with other active agents, including antibodies, alkylating agents, angiogenesis inhibitors, antimetabolites, DNA cleavers, DNA crosslinkers, DNA intercalators, DNA minor groove binders, enediynes, heat shock protein 90 inhibitors, histone deacetylase inhibitors, immunomodulators, microtubule stabilizers, nucleoside (purine or pyrimidine) analogs, nuclear export inhibitors, proteasome inhibitors, topoisomerase (I or II) inhibitors, tyrosine kinase inhibitors, and serine/threonine kinase inhibitors. Specific therapeutic agents include adalimumab, ansamitocin P3, auristatin, bendamustine, bevacizumab, bicalutamide, bleomycin, bortezomib, busulfan, callistatin A, camptothecin, capecitabine, carboplatin, carmustine, cetuximab, cisplatin, cladribin, cytarabin, cryptophycins, dacarbazine, dasatinib, daunorubicin, docetaxel, doxorubicin, duocarmycin, dynemycin A, epothilones, etoposide, floxuridine, fludarabine, 5-fluorouracil, gefitinib, gemcitabine, ipilimumab, hydroxyurea, imatinib, infliximab, interferons, interleukins, beta-lapachone, lenalidomide, irinotecan, maytansine, mechlorethamine, melphalan, 6-mercaptopurine, methotrexate, mitomycin C, nilotinib, oxaliplatin, paclitaxel, procarbazine, suberoylanilide hydroxamic acid (SAHA), 6-thioguanidine, thiotepa, teniposide, topotecan, trastuzumab, trichostatin A, vinblastine, vincristine, and vindesine.

### VII. Therapeutic Uses of the Double Payload ADCs

Another aspect of the disclosure relates to a method of using the antibody-drug conjugates, and pharmaceutical compositions thereof for treating cancer. In certain embodiments, the conjugates provide conjugation-specific tumor or cancer drug targeting, thus reducing general toxicity of the compounds of the disclosure. In another embodiment, the antibody moiety binds to the tumor cell or cancer cell. In another embodiment, the antibody moiety binds to a tumor cell or cancer cell antigen which is on the surface of the tumor cell or cancer cell. In another embodiment, the antibody moiety binds to a tumor cell or cancer cell antigen which is an extracellular matrix protein associated with the tumor cell or cancer cell. The specificity of the antibody moiety for a particular tumor cell or cancer cell can be important for determining those tumors or cancers that are most effectively treated.

Particular types of cancers that can be treated with an ADC of the disclosure include but are not limited to, carcinomas of the bladder, breast, cervix, colon, endometrium, kidney, lung, esophagus, ovary, prostate, pancreas, skin, stomach, and testes; and blood born cancers including but not limited to leukemias and lymphomas.

In one aspect, an antibody-drug conjugate provided herein is used in a method of inhibiting proliferation of a cancer cell, the method comprising exposing the cell to the antibody-drug conjugate under conditions permissive for binding of the antibody or antibody-drug conjugates to a tumor-associated antigen on the surface of the cell, thereby inhibiting the proliferation of the cell. In certain embodiments, the method is an in vitro or an in vivo method. In further embodiments, the cell is a lymphocyte, lymphoblast, monocyte, or myelomonocyte cell.

In another aspect, an ADC for use as a medicament is provided. In further aspects, an ADC for use in a method of treatment is provided. In certain embodiments, an ADC for use in treating cancer is provided. In certain embodiments, the disclosure provides an ADC for use in a method of treating an individual comprising administering to the individual an effective amount of the ADC. In one such embodiment, the method further comprises administering to the individual an effective amount of at least one additional therapeutic agent, e.g., as described herein.

In a further aspect, the disclosure provides for the use of an ADC in the manufacture or preparation of a medicament. In one embodiment, the medicament is for treatment of cancer, the method comprising administering to an individual having cancer an effective amount of the medicament. In one such embodiment, the method further comprises administering to the individual an effective amount of at least one additional therapeutic agent, e.g., as described herein.

In a further aspect, the disclosure provides a method for treating cancer. In one embodiment, the method comprises administering to an individual having such cancer, characterized by detection of a tumor-associated expressing antigen, an effective amount of an antibody-drug conjugate of the disclosure. In one such embodiment, the method further comprises administering to the individual an effective amount of at least one additional therapeutic agent.

ADCs of the disclosure can be used either alone or in combination with other agents in a therapy. For instance, an ADC of the disclosure may be co-administered with at least one additional therapeutic agent, such as a chemotherapeutic agent.

Such combination therapies noted herein encompass combined administration (where two or more therapeutic agents are included in the same or separate formulations), and separate administration, in which case, administration of the ADC of the disclosure can occur prior to, simultaneously, and/or following, administration of the additional therapeutic agent and/or adjuvant. ADCs of the disclosure can also be used in combination with radiation therapy.

ADCs of the disclosure (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The following examples are offered by way of illustration and not by way of limitation.

### EXAMPLES

### I. Synthetic Experimental Procedures

Experiments are generally carried out under inert atmosphere (nitrogen or argon), particularly in cases where oxygen- or moisture-sensitive reagents or intermediates are employed. Commercial solvents and reagents are generally used without further purification, including anhydrous solvents where appropriate. Mass spectrometry data is reported from either liquid chromatography-mass spectrometry (LCMS) or atmospheric pressure chemical ionization (APCI). Chemical shifts for nuclear magnetic resonance (NMR) data are expressed in parts per million (ppm, δ) referenced to residual peaks from the deuterated solvents employed.

In general, reactions are followed by thin layer chromatography, LCMS or HPLC, and subjected to work-up when appropriate. Purifications may vary between experiments: in general, solvents and the solvent ratios used for eluents/gradients are chosen to provide appropriate retention times. Unless otherwise specified, reverse phase HPLC fractions are concentrated via lyophilization/freeze-drying. Intermediate and final compounds are stored at (0° C.) or room temperature in closed vials or flasks under nitrogen.

### Example 1

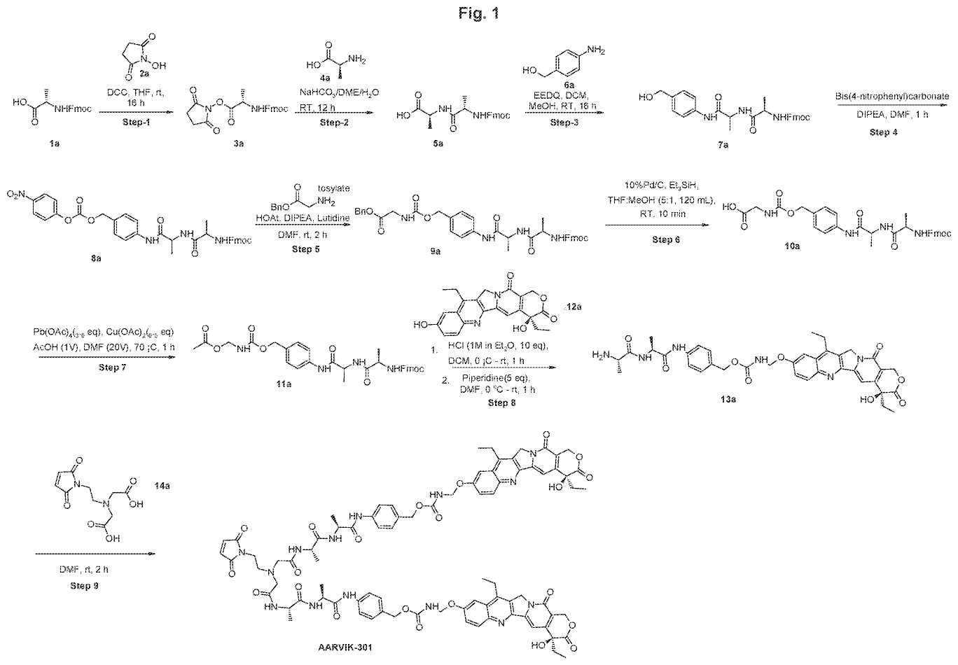

Compound AARVIK-301 is synthesized according to the scheme depicted in FIG. 1.

### Example 2

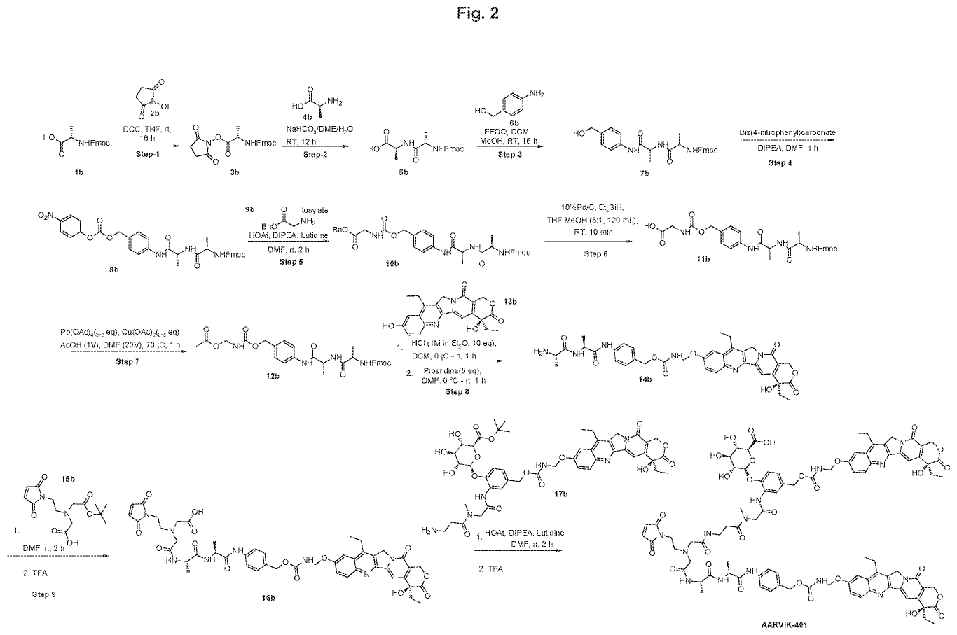

Compound AARVIK-401 is synthesized according to the scheme depicted in FIG. 2.

### Example 3

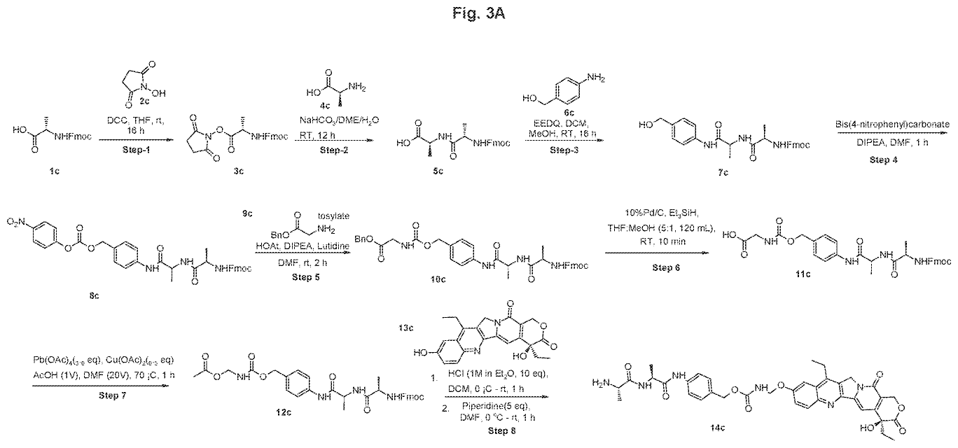

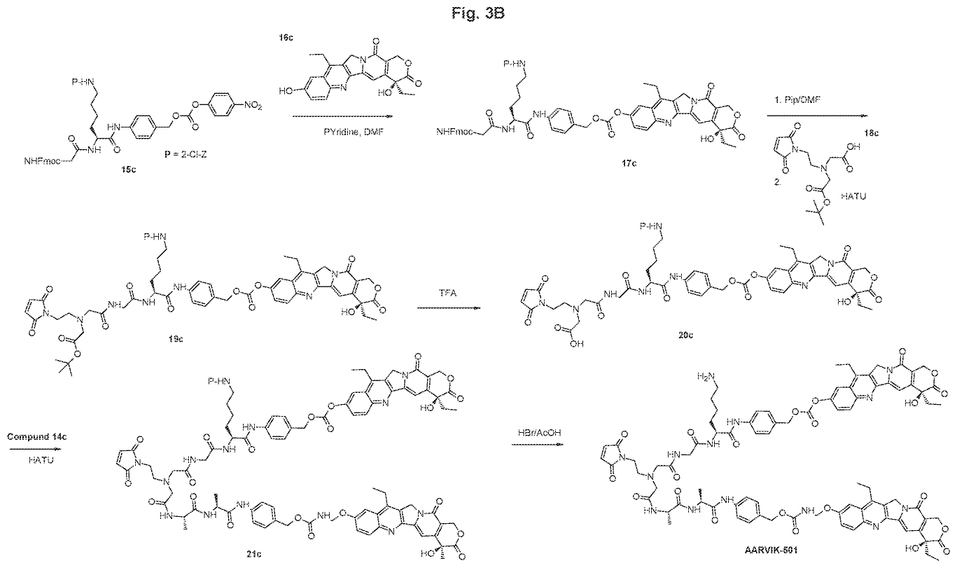

Compound AARVIK-501 is synthesized according to the scheme depicted in FIG. 3.

### Example 4

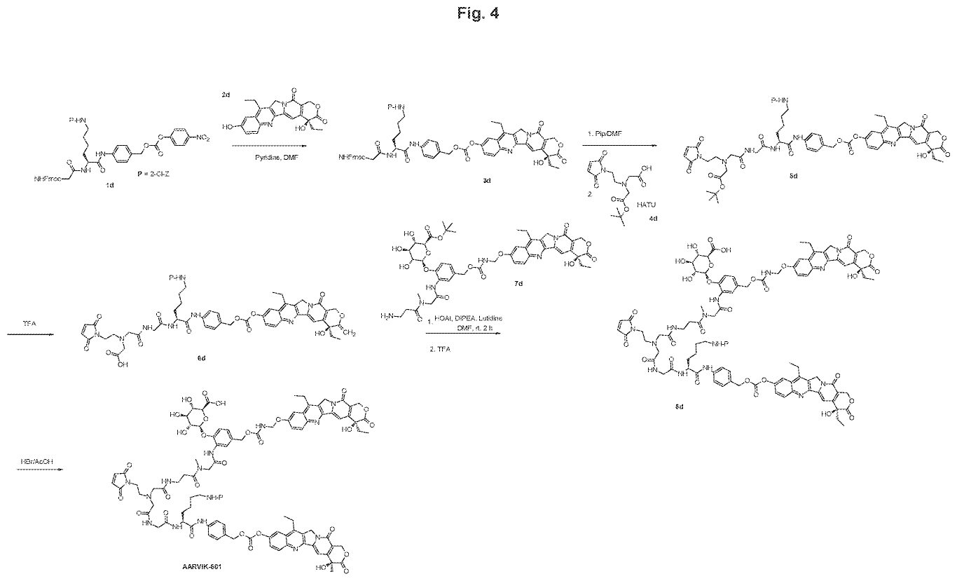

Compound AARVIK-501 is synthesized according to the scheme depicted in FIG. 4.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality” and “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “about” refers to a range that is 5% plus or minus from a stated numerical value within the context of the particular usage. So, for example, “about 100” means between 95 and 105. The term “consisting essentially of” refers to the inclusion of recited elements and other elements that do not materially affect the basic and novel characteristics of a claimed combination.

It should be understood that the description and the drawings are not intended to limit the disclosure to the particular form disclosed, but to the contrary, the intention is to cover all modifications, equivalents, and alternatives falling within the spirit and scope of the present disclosure as defined by the appended claims. Further modifications and alternative embodiments of various aspects of the disclosure will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims. Headings used herein are for organizational purposes only and are not meant to be used to limit the scope of the description.

All publications, patents, and patent applications mentioned in this specification are herein incorporated by reference to the same extent as if each individual publication, patent, or patent application was specifically and individually indicated to be incorporated by reference.

What is claimed is:

1. A compound of Formula (I): 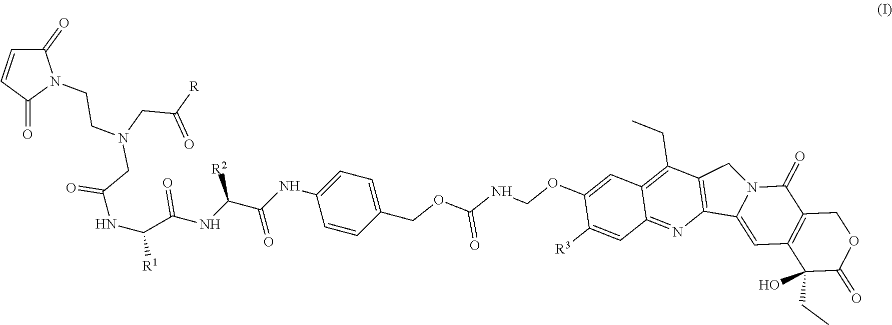
wherein:
R is selected from the group consisting of Formula (II), Formula (III), and Formula (IV): 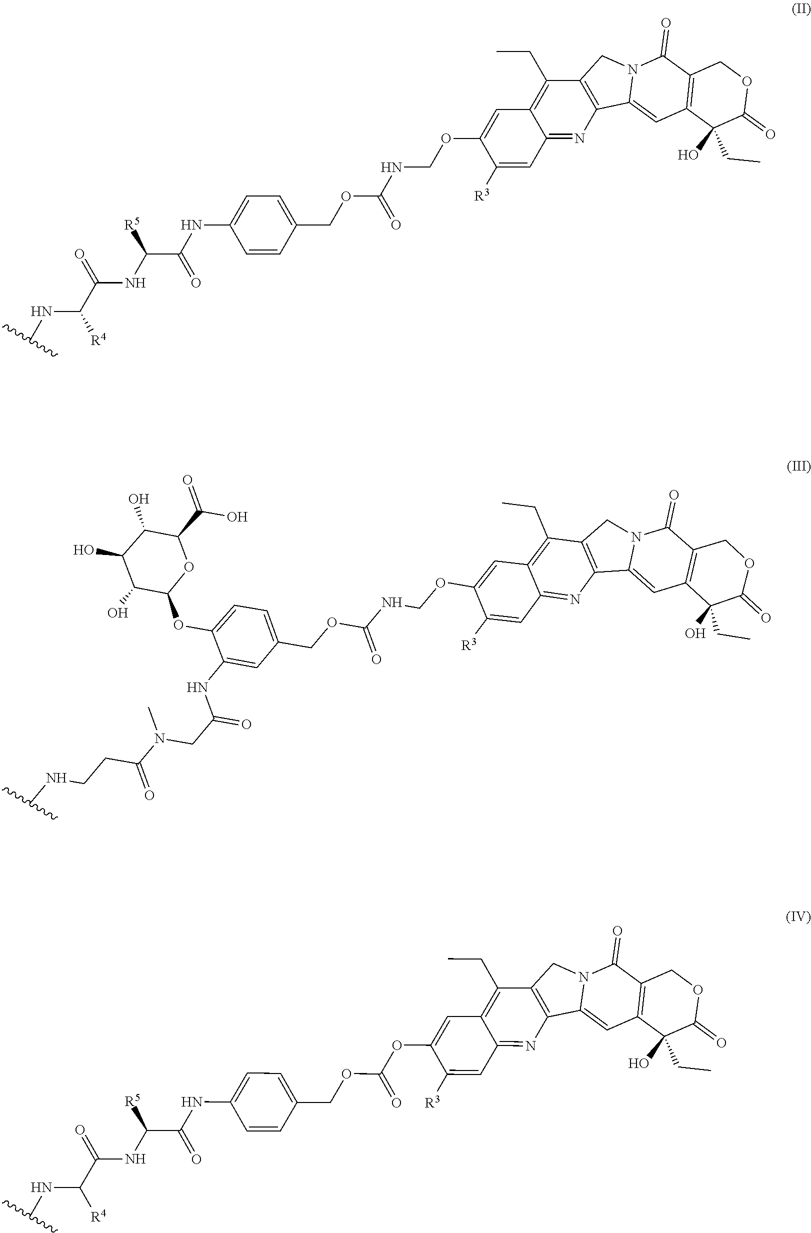
wherein:
R¹ is —CH₃ or —CH(CH₃)₂;
R² is —CH₃, —(CH₂)₃NHC(═O)NH₂ or —CH(CH₃)₂;
R³ is independently selected from H and F;
R⁴ is —H, —CH₃ or —CH(CH₃)₂; and
R⁵ is —CH₃, —(CH₂)₄NH₂, —(CH₂)₃NHC(═O)NH₂ or —CH(CH₃)₂;
or a pharmaceutically acceptable salt thereof.

2. The compound of claim 1, selected from the group consisting of:

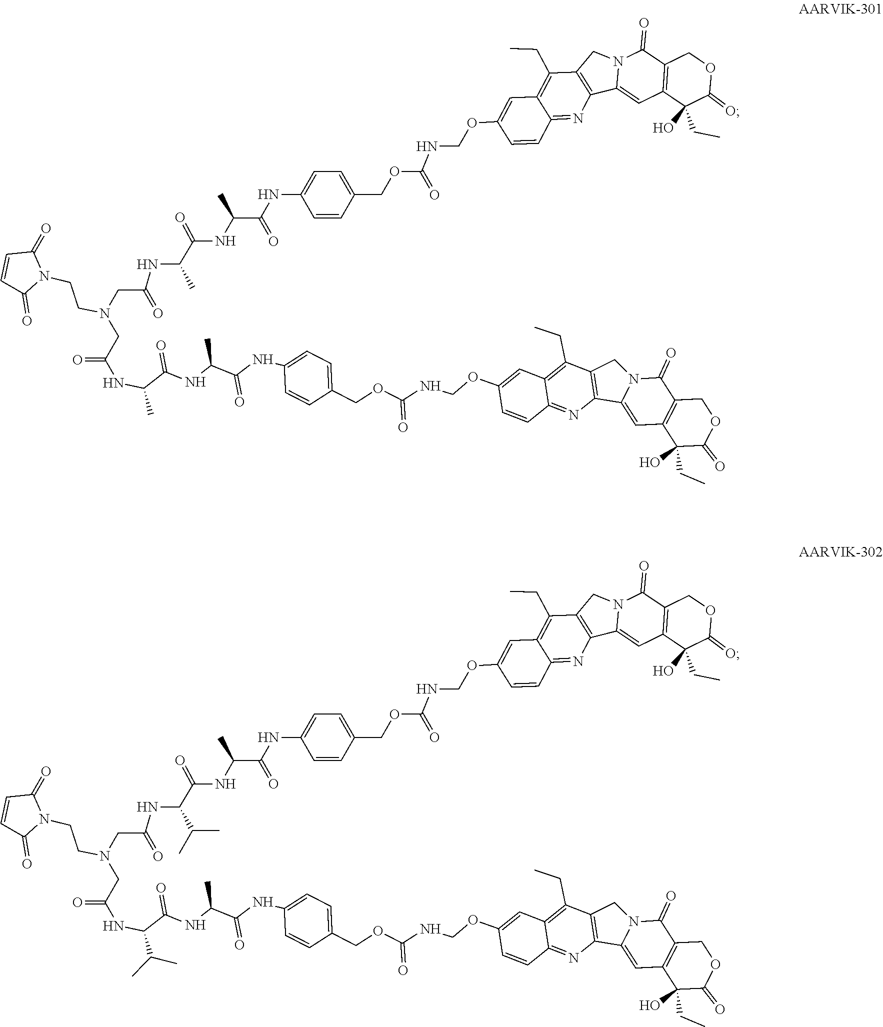 [SMILES: CCc1c2c(nc3ccc(OCNC(=O)OCc4ccc(NC(=O)[C@H](C)NC(=O)[C@H](C)NC(=O)CN(CCN5C(=O)C=CC5=O)CC(=O)N[C@@H](C)C(=O)N[C@@H](C)C(=O)Nc5ccc(COC(=O)NCOc6ccc7nc8c(c(CC)c7c6)Cn6c-8cc7c(c6=O)COC(=O)[C@]7(O)CC)cc5)cc4)cc13)-c1cc3c(c(=O)n1C2)COC(=O)[C@]3(O)CC ; CCc1c2c(nc3ccc(OCNC(=O)OCc4ccc(NC(=O)[C@H](C)NC(=O)[C@@H](NC(=O)CN(CCN5C(=O)C=CC5=O)CC(=O)N[C@H](C(=O)N[C@@H](C)C(=O)Nc5ccc(COC(=O)NCOc6ccc7nc8c(c(CC)c7c6)Cn6c-8cc7c(c6=O)COC(=O)[C@]7(O)CC)cc5)C(C)C)C(C)C)cc4)cc13)-c1cc3c(c(=O)n1C2)COC(=O)[C@]3(O)CC]

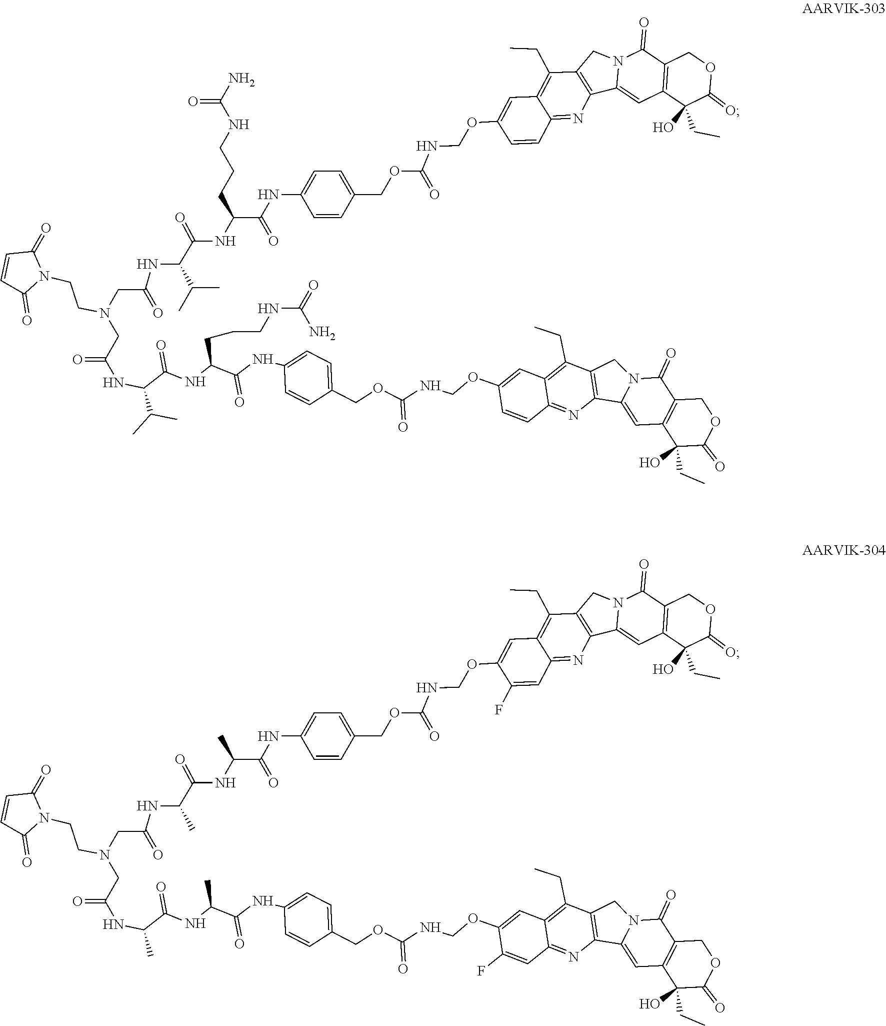

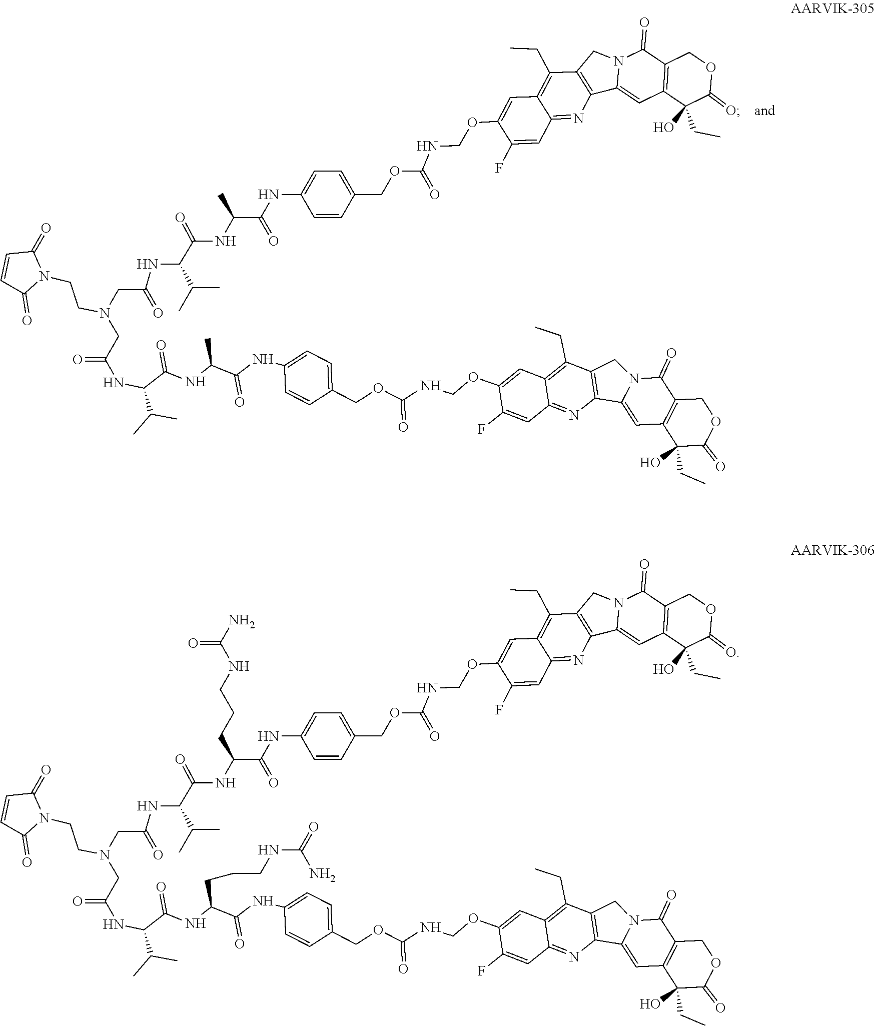

3. The compound of claim 1, selected from the group consisting of:

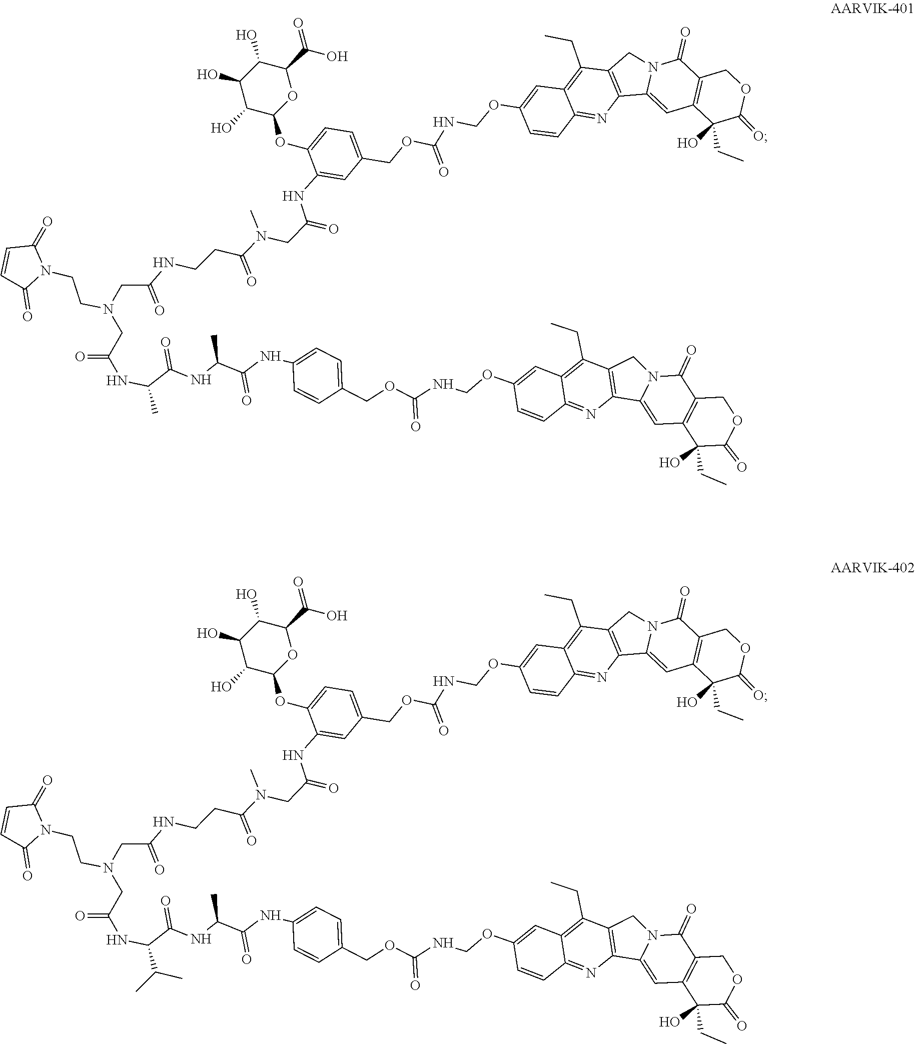

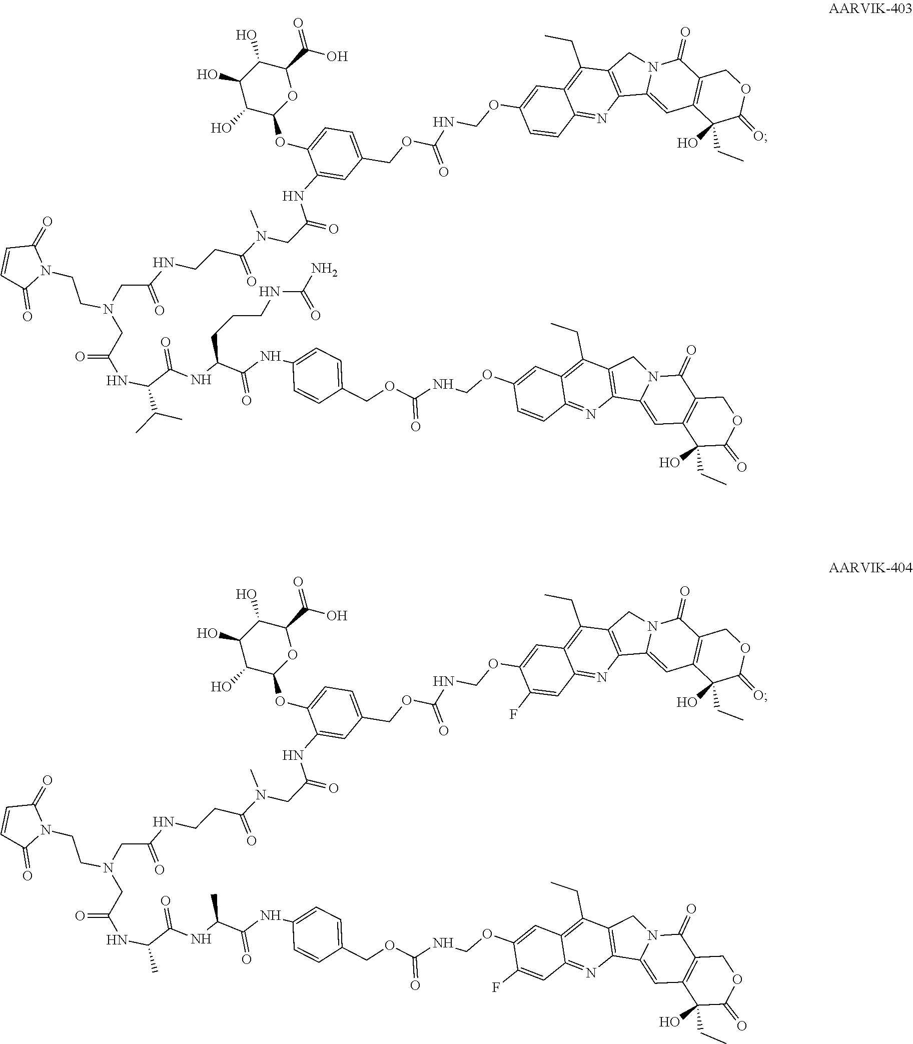

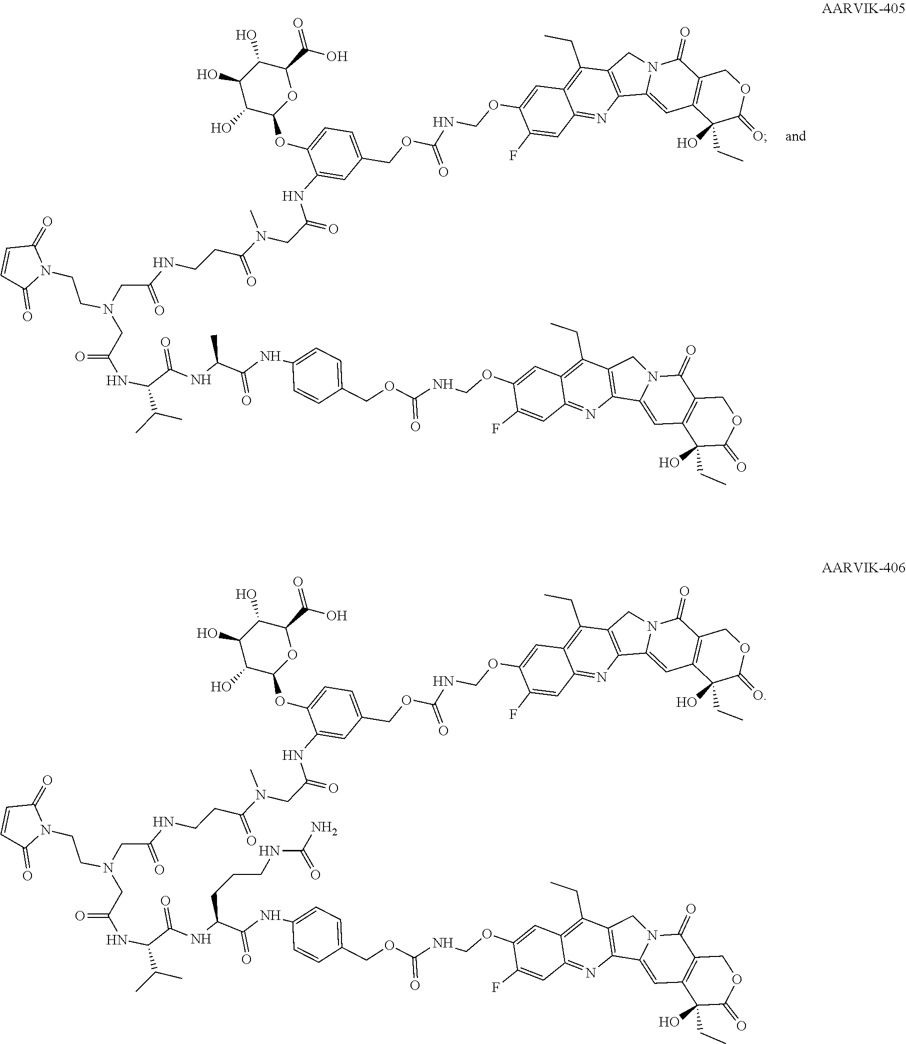

4. The compound of claim 1, selected from the group consisting of:

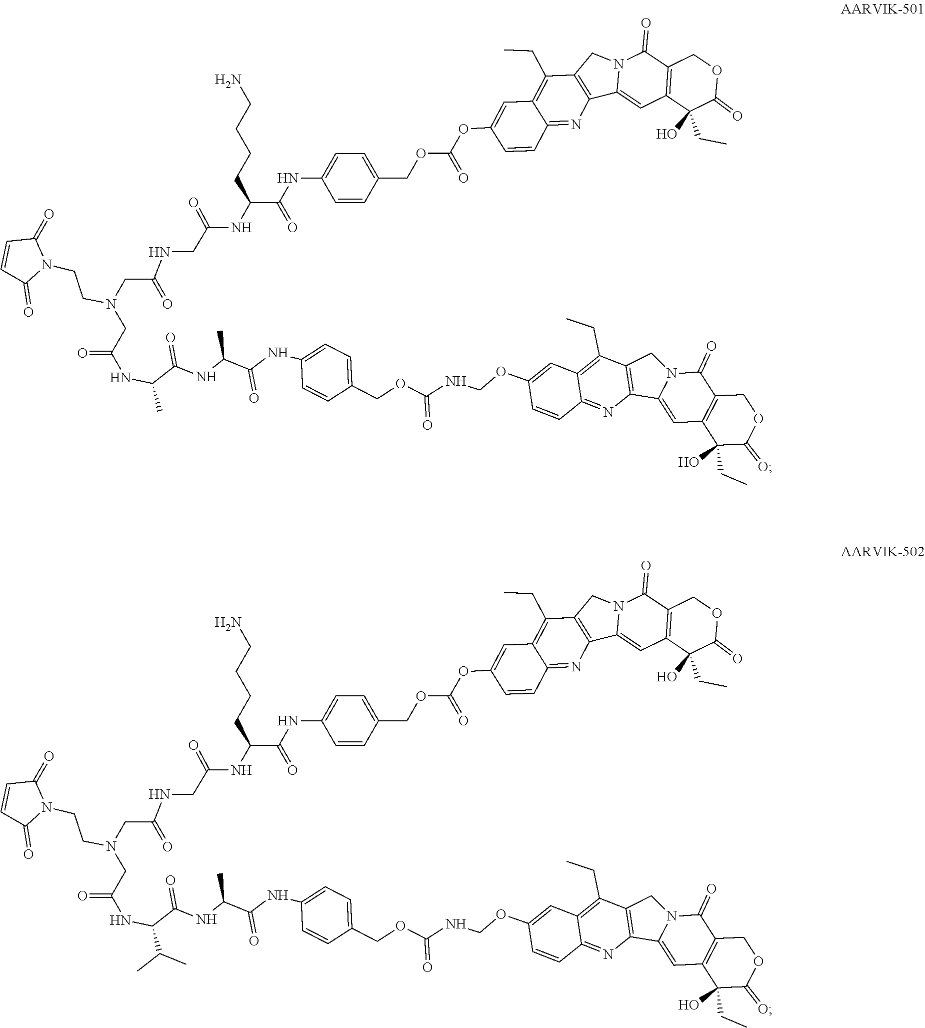 [SMILES: CCc1c2c(nc3ccc(OCNC(=O)OCc4ccc(NC(=O)[C@H](C)NC(=O)[C@H](C)NC(=O)CN(CCN5C(=O)C=CC5=O)CC(=O)NCC(=O)N[C@@H](CCCCN)C(=O)Nc5ccc(COC(=O)Oc6ccc7nc8c(c(CC)c7c6)Cn6c-8cc7c(c6=O)COC(=O)[C@]7(O)CC)cc5)cc4)cc13)-c1cc3c(c(=O)n1C2)COC(=O)[C@]3(O)CC ; CCc1c2c(nc3ccc(OCNC(=O)OCc4ccc(NC(=O)[C@H](C)NC(=O)[C@@H](NC(=O)CN(CCN5C(=O)C=CC5=O)CC(=O)NCC(=O)N[C@@H](CCCCN)C(=O)Nc5ccc(COC(=O)Oc6ccc7nc8c(c(CC)c7c6)Cn6c-8cc7c(c6=O)COC(=O)[C@]7(O)CC)cc5)C(C)C)cc4)cc13)-c1cc3c(c(=O)n1C2)COC(=O)[C@]3(O)CC]

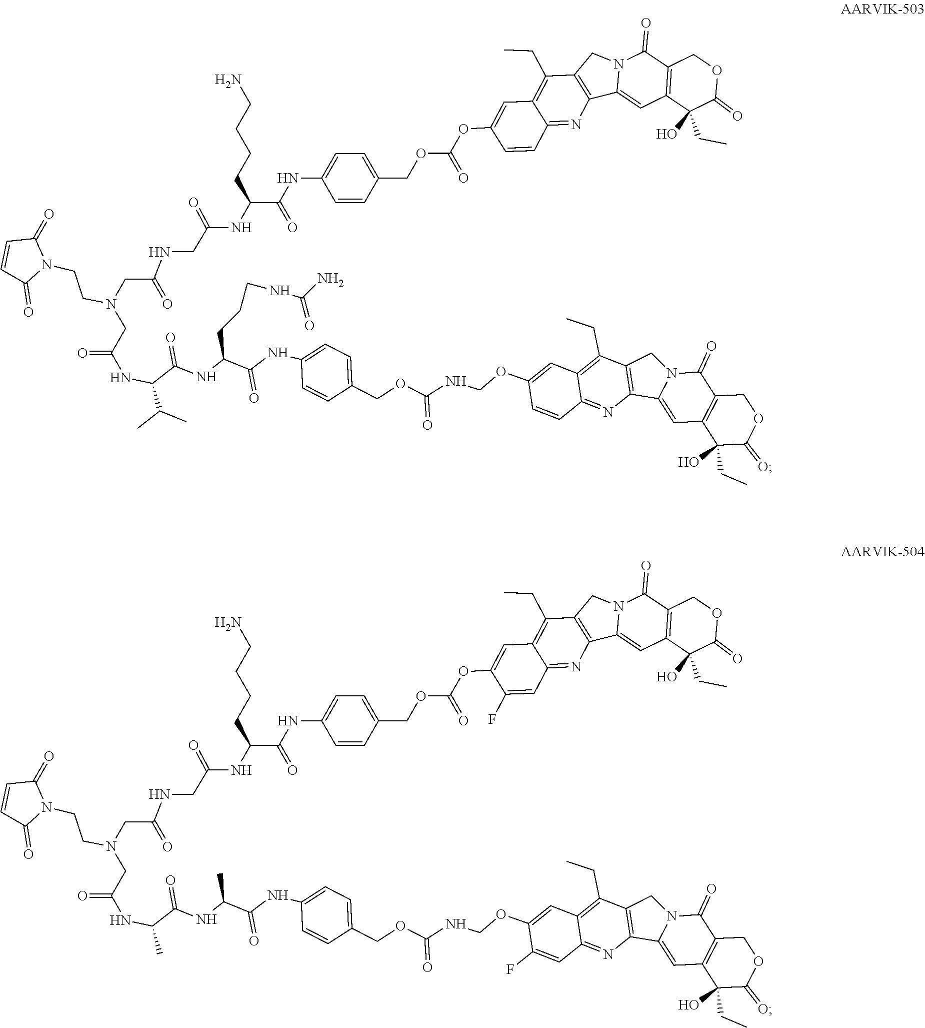 [SMILES: CCc1c2c(nc3ccc(OCNC(=O)OCc4ccc(NC(=O)[C@H](CCCNC(N)=O)NC(=O)[C@@H](NC(=O)CN(CCN5C(=O)C=CC5=O)CC(=O)NCC(=O)N[C@@H](CCCCN)C(=O)Nc5ccc(COC(=O)Oc6ccc7nc8c(c(CC)c7c6)Cn6c-8cc7c(c6=O)COC(=O)[C@]7(O)CC)cc5)C(C)C)cc4)cc13)-c1cc3c(c(=O)n1C2)COC(=O)[C@]3(O)CC ; CCc1c2c(nc3cc(F)c(OCNC(=O)OCc4ccc(NC(=O)[C@H](C)NC(=O)[C@H](C)NC(=O)CN(CCN5C(=O)C=CC5=O)CC(=O)NCC(=O)N[C@@H](CCCCN)C(=O)Nc5ccc(COC(=O)Oc6cc7c(CC)c8c(nc7cc6F)-c6cc7c(c(=O)n6C8)COC(=O)[C@]7(O)CC)cc5)cc4)cc13)-c1cc3c(c(=O)n1C2)COC(=O)[C@]3(O)CC]

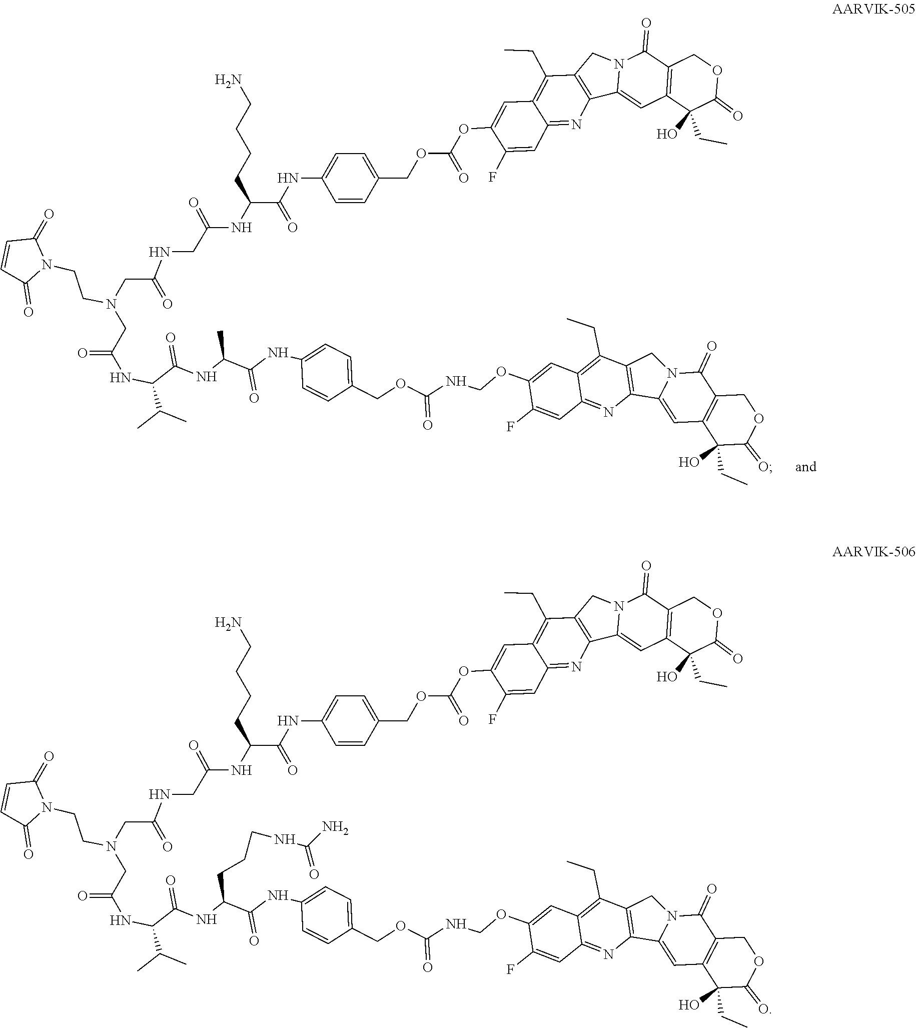

5. A compound of Formula (V): 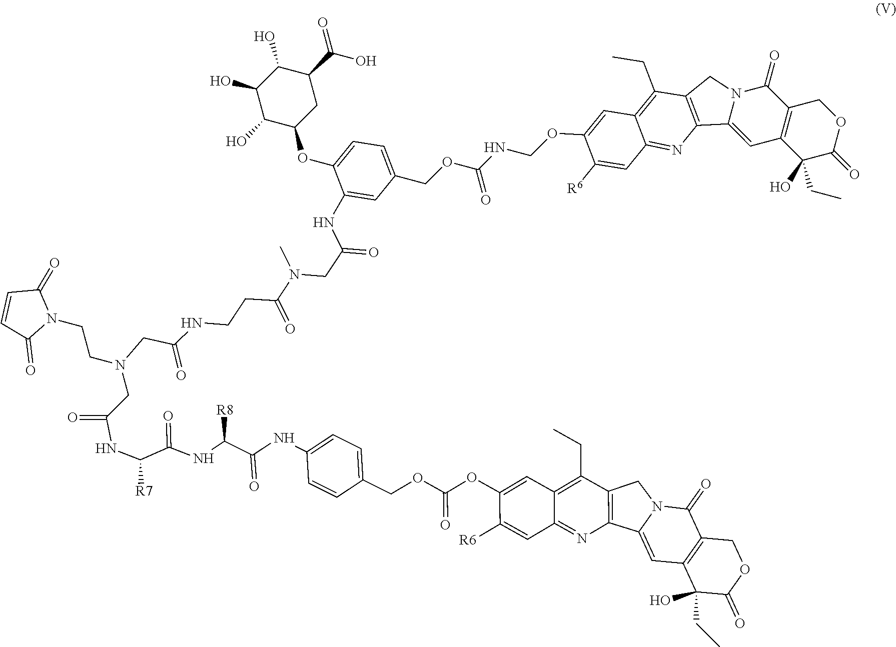
wherein:
R⁶ is H or F;
R⁷ is —H, —CH₃ or —CH(CH₃)₂;
R⁸ is —CH₃, —(CH₂)₄NH₂, —(CH₂)₃NHC(═O)NH₂ or —CH(CH₃)₂;
or a pharmaceutically acceptable salt thereof.

6. The compound of claim 5, selected from the group consisting of:

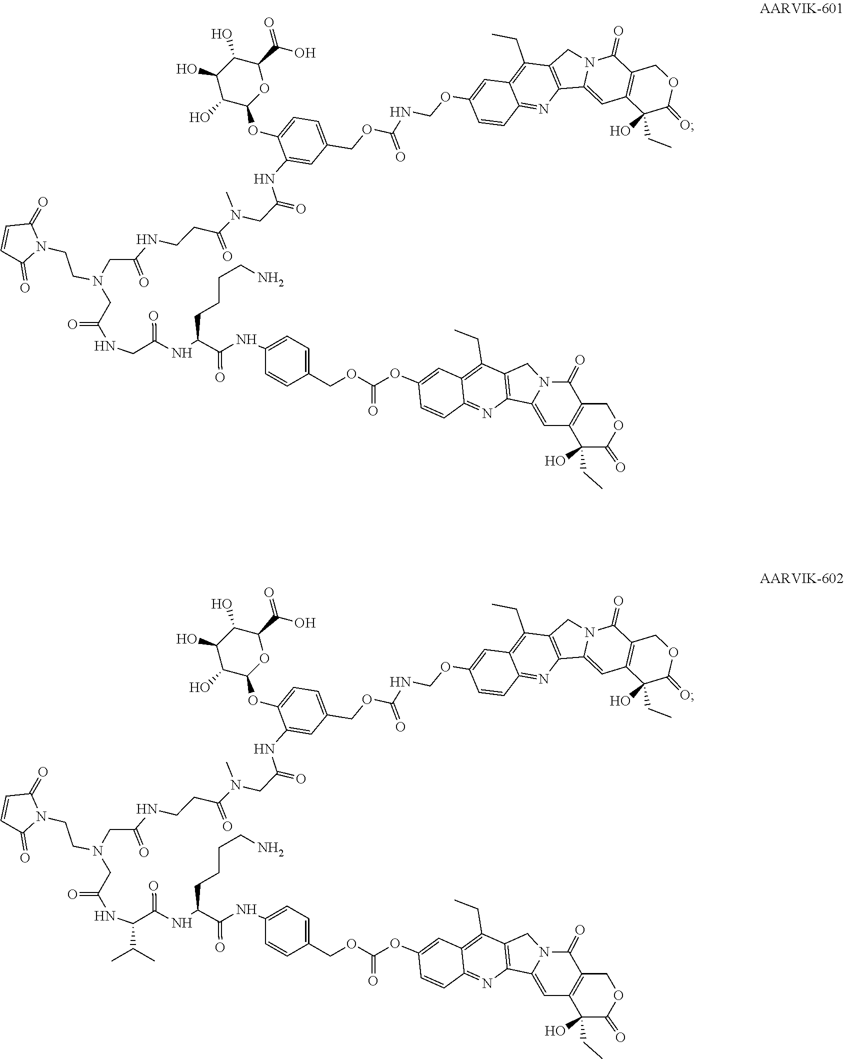

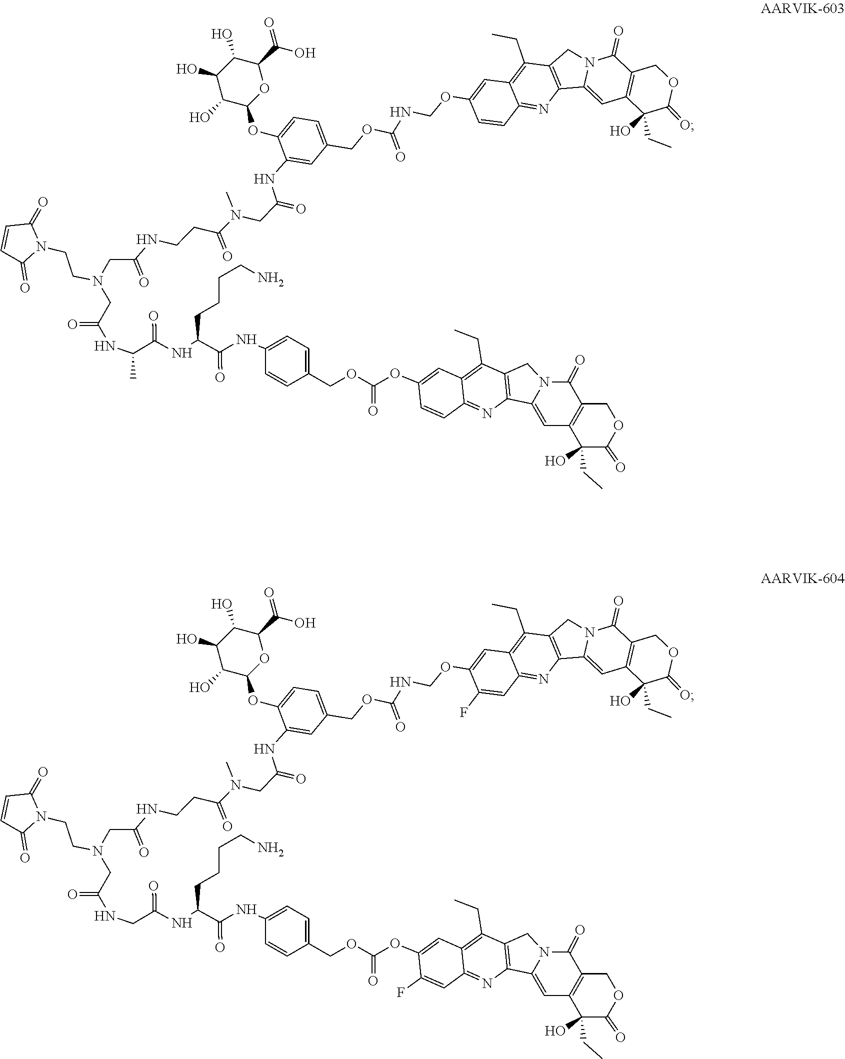

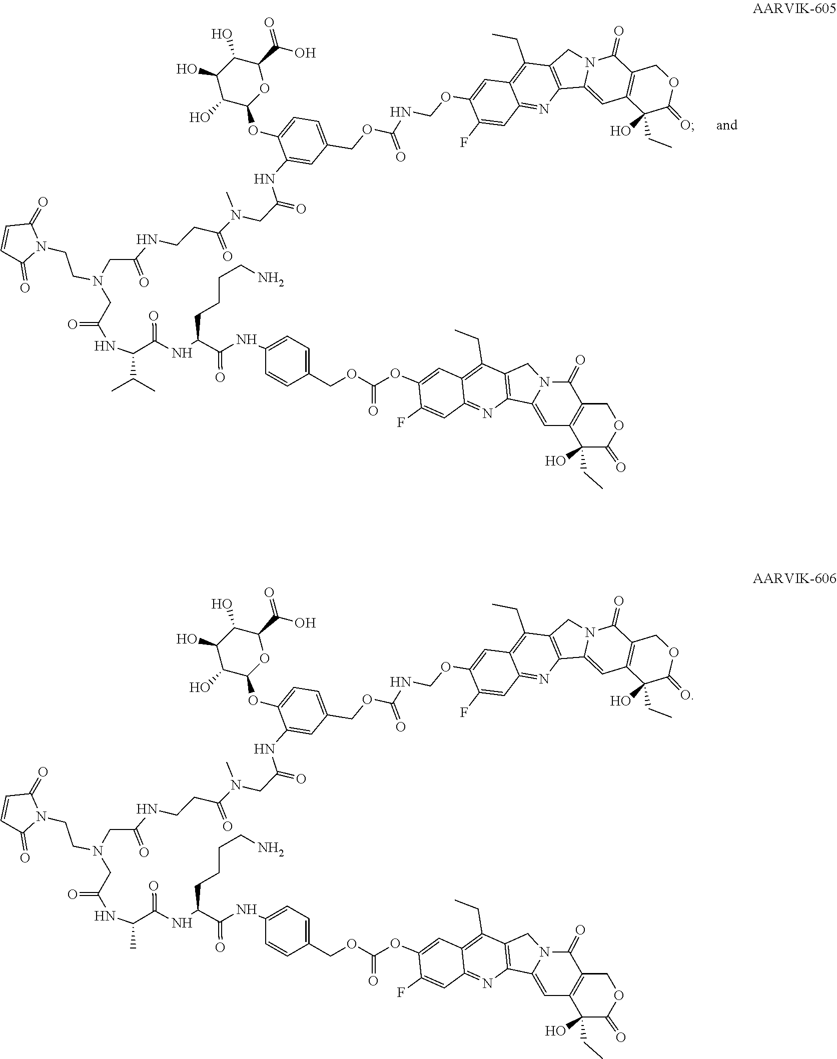

7. A compound of Formula (VI): 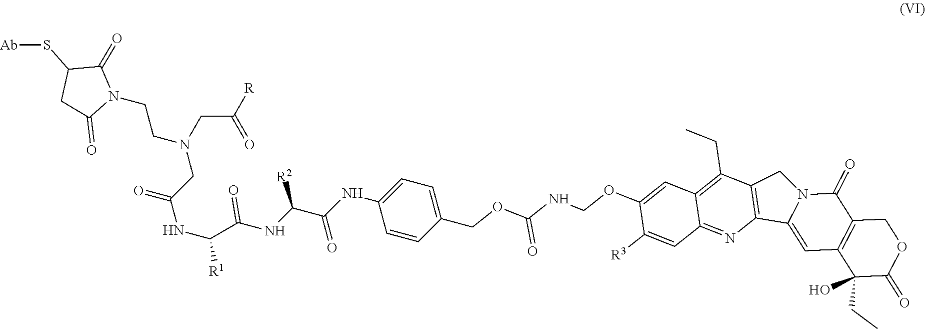
wherein Ab is an antibody; and
R is selected from the group consisting of Formula (II), Formula (III), and Formula (IV): 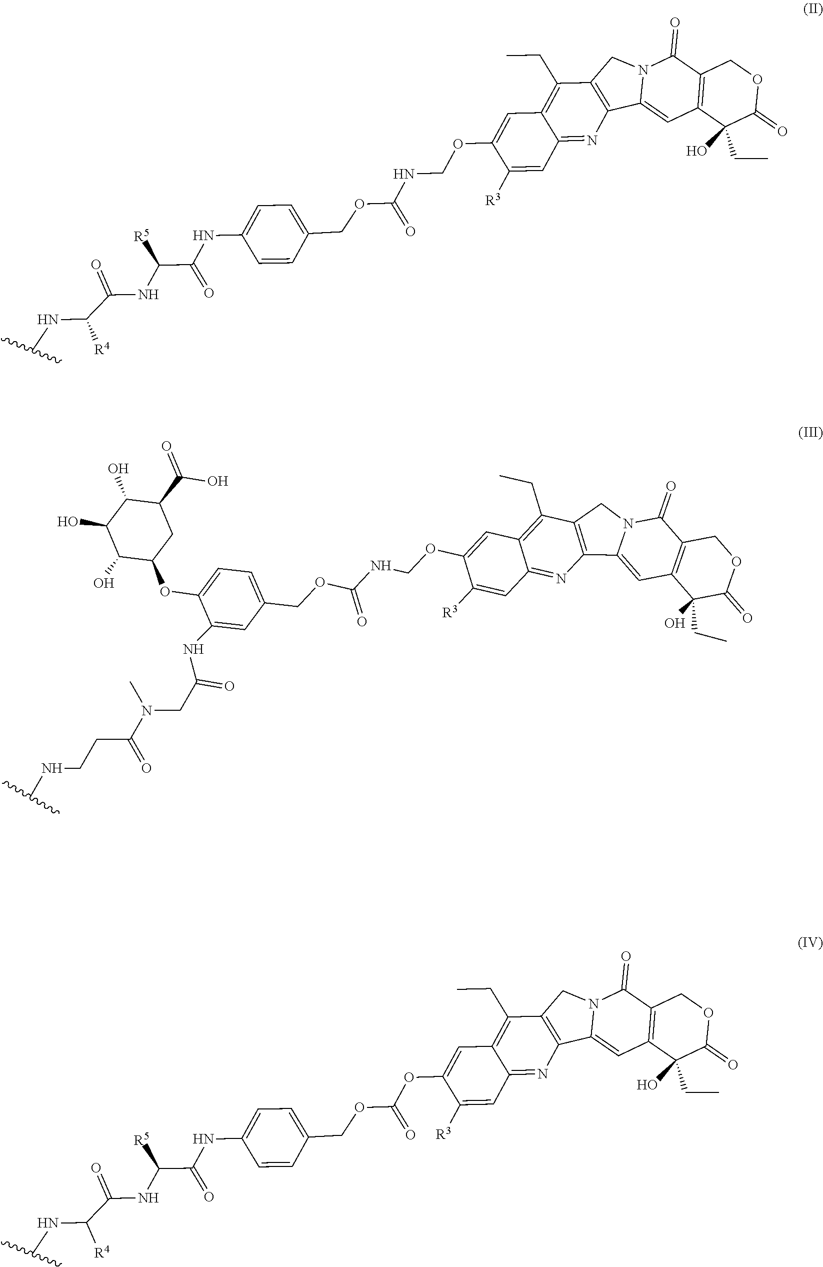
R¹ is —CH₃ or —CH(CH₃)₂;
R² is —CH₃, —(CH₂)₃NHC(═O)NH₂ or —CH(CH₃)₂;
R³ is independently selected from H and F;
R⁴ is —H, —CH₃ or —CH(CH₃)₂;
R⁵ is —CH₃, —(CH₂)₄NH₂, —(CH₂)₃NHC(═O)NH₂ or —CH(CH₃)₂;
or a pharmaceutically acceptable salt thereof.

8. A compound of Formula (VII) 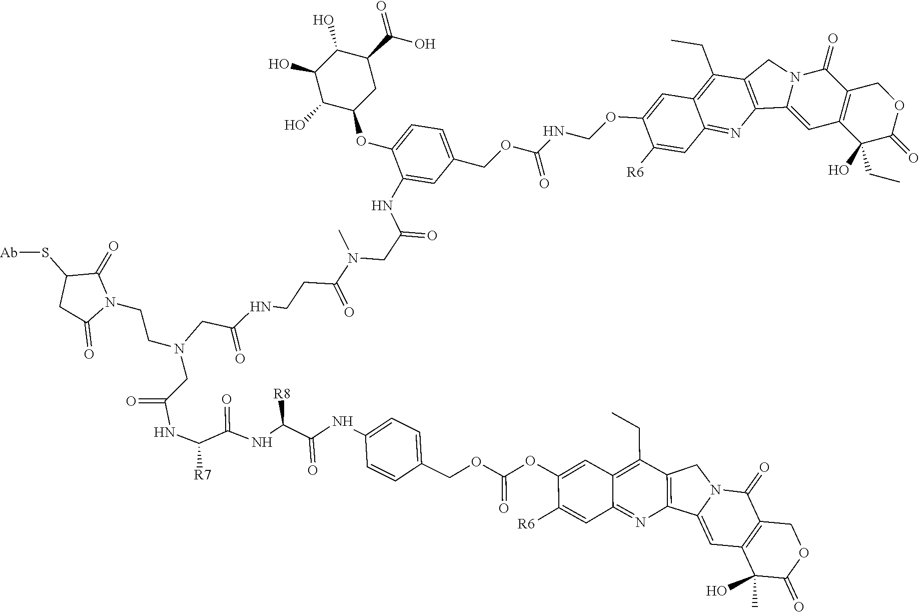
wherein Ab is an antibody; and
R⁶ is H or F;
R⁷ is —H, —CH₃ or —CH(CH₃)₂;
R⁸ is —CH₃, —(CH₂)₄NH₂, —(CH₂)₃NHC(═O)NH₂ or —CH(CH₃)₂;
or a pharmaceutically acceptable salt thereof.

9. A pharmaceutical composition comprising a therapeutically effective amount of the compound or salt of claim of claim 1, or an immunoconjugate thereof, and a pharmaceutically acceptable carrier.

10. A method of for the treatment of cancer, comprising administering a pharmaceutically acceptable amount of the pharmaceutical composition of claim 9.

11. A kit comprising:
a) a compound of claim 1 or an immunoconjugate thereof; and
b) instructions for use.

12. A method comprising combining a compound of claim 1 and an antibody and providing conditions such that the compound conjugates to produce a drug-antibody conjugate.

13. A method comprising contacting a cell with a compound of claim 1 or an immunoconjugate thereof.

14. A composition comprising a cell and a compound of claim 1 or an immunoconjugate thereof.

15. Use of a compound of claim 1 in the manufacture of a medicament.

16. Use of a compound of claim 1 to produce an antibody-drug conjugate.
